(19) United States
(12) Patent Application Publication
(10) Pub. No.: US 2020/0331984 A1
(43) Pub. Date: Oct. 22, 2020

(54) HARNESSING PROTEIN-BASED DRUGS COMPRISING AN ANCHOR DOMAIN FOR USE ON THE OCULAR SURFACE

(71) Applicant: UNIVERSITY OF PITTSBURGH - OF THE COMMONWEALTH SYSTEM OF HIGHER EDUCATION, Pittsburgh, PA (US)

(72) Inventors: Robert Michael Queen Shanks, Pittsburgh, PA (US); Jes K. Klarlund, Pittsburgh, PA (US)

(21) Appl. No.: 16/334,691

(22) PCT Filed: Sep. 19, 2017
(86) PCT No.: PCT/US17/52288

§ 371 Date: Mar. 19, 2019

Related U.S. Application Data
(60) Provisional application No. 62/396,958, filed on Sep. 20, 2016.

(51) Int. Cl. C07K 14/755; C07K 14/42; C07K 14/33; C07K 16/22; C07K 16/24; C07K 16/42; A61P 27/02

(52) U.S. Cl. CPC C07K 14/755; C07K 14/42; C07K 14/33; C07K 16/22; A61K 38/00; C07K 16/241; C07K 16/4208; A61P 27/02; C07K 16/248

(57) ABSTRACT

Pharmaceutical compositions are disclosed including an effective amount of a fusion protein that contains an anchor domain and a therapeutic polypeptide for use in treating a disorder that affects the eye (e.g., corneal haze or scarring, dry eye disease, and inflammation of an ocular surface). Fusion proteins are disclosed that includes an anchor domain and a therapeutic polypeptide. Some embodiments include an isolated nucleic acid molecule that can encode the fusion protein. Methods are also disclosed for treating a subject with a disorder that affects the eye (e.g., corneal haze or scarring, dry eye disease, and inflammation of an ocular surface) that includes administering to the eye of the subject a therapeutically effective amount of the fusion protein disclosed herein.

### CROSS REFERENCE TO RELATED APPLICATIONS

This application claims the benefit of U.S. Provisional Application No. 62/396,958, filed Sep. 20, 2016, which is herein incorporated by reference in its entirety.

### FIELD

This relates to the field of protein-based drugs for use for treating disorders of the eye, such as corneal haze and scarring, dry eye disease, and ocular inflammation.

### ACKNOWLEDGMENT OF GOVERNMENT SUPPORT

This invention was made with government support under grant nos. AI085570 awarded by the National Institutes of Health. The government has certain rights in the invention.

### BACKGROUND

Diseases of the surface of the eye are common. Corneal haze reduces or abolishes vision, is a serious complication of many eye diseases and surgical interventions, and is a leading cause of blindness world-wide (Bourne et al., Lancet Glob Health, 1:e339-349, 2013; Oliva et al., Indian J Ophthalmol, 60:423-427, 2012). Persistent corneal haze is most commonly caused by excessive activity of tumor growth factor-β (TGFβ), which stimulates transformation of keratocytes into myofibroblasts that synthetize large amounts of disorganized extracellular matrix, thereby destroying the normal structure and transparency of the cornea (Torricelli et al., Exp Eye Res, 142:110-118, 2016; Tomas-Juan et al., J Optom 8, 149-169, 2015; Hinz Exp Eye Res, 142:56-70, 2016). Therefore, there has been much interest in blocking the activity of TGFβ. Although several blocking agents exist (Finnson et al., Adv Wound Care (New Rochelle) 2:195-214, 2013) none are presently used to treat or prevent haze in corneas.

Dry eye affects up to one-third of the adult population and can vary in intensity from mildly annoying to interfering strongly with daily life (Stapleton et al., Ocul Surf, 15:334-365, 2017). Over-the-counter drops are popular, but there is little evidence that they have any actual pharmacological effects (Pucker et al., Cochrane Database of Systematic Reviews, 2, 2016). Dry eye is fundamentally an inflammatory disease (Bron et al., Ocul Surf, 15:438-510, 2017), and a great number of biological reagents could be used to treat the condition (O'Shea et al., Cell, 157:227-240, 2014). However, using these reagents has been precluded by the lack of an effective delivery method to the eye surface.

Other corneal diseases commonly cause blindness. There have been tremendous advances in pharmacological science recently by development of biological drugs (Andrews, L. et al., Human and Exp. Toxocol. 34:1279-1285, 2015; O'Shea et al, Cell, 157: 227-240, 2014), and they are now used to treat rheumatoid arthritis, psoriasis, some forms of cancer, and other conditions. Many of these drugs could be extremely useful to manage diseases of the ocular surface. However, the problem is delivery; the clearing action of the tear film combined with blinking removes therapeutics within 2-5 minutes; most drugs are removed too quickly to have any significant effects when they are applied in eye drops (Kim, Y.C. et al., J. Controlled release, 190,172-181, 2014).

A great variety of experimental ocular drug delivery systems are under development, which is not surprising given the importance of drug delivery to the eye (Kim, Y. C. et al., J Control Release, 190:172-181, 2014; Souza, J. G. et al., J Pharm Pharmacol, 66:507-530, 2014). The limitations of previous studies are that delivery depends on some form of carrier-based device. The manufacture of many of these requires harsh conditions involving organic solvents, and most are only suited to deliver small molecular weight compounds. Systems proposed to deliver proteins (Vaishya, R. et al., Expert Opin Drug Deliv, 12:415-440, 2015; Agarwal, P. et al., Drug Discov Today, 18:337-349, 2013) include various forms of gels and pellets or permeable contact lenses, which are cumbersome and/or interfere with vision and are rarely used in practice. Thus, the need exists for effective drug delivery to the ocular surface, particularly such that vision is not disrupted.

### SUMMARY

Disclosed herein are fusion proteins that include an anchor domain and a therapeutic polypeptide, which can be used to treat a disorder that affects the eye, such as, but not limited to, corneal haze and scarring, dry eye disease, and ocular inflammation. In some embodiments, the fusion proteins can include an anchor domain, such as a lectin carbohydrate-binding polypeptide, a von Willebrand factor (vWF) collagen-binding polypeptide, a Clostridium collagenase (ColH) collagen-binding polypeptide, or a heparin-binding (HS) polypeptide, and the therapeutic polypeptide can include a transforming growth factor beta 1 (TGFβ) antagonist, a tumor necrosis factor alpha (TNFα) antagonist, an interleukin (IL)-1β antagonist, an IL-6 antagonist, an immunoglobulin-binding (Ig) polypeptide, or an interferon (IFN)γ antagonist. In other embodiments, the fusion proteins can include an anchor domain, such as a lectin carbohydrate-binding polypeptide, a von Willebrand factor (vWF) collagen-binding polypeptide, or a Clostridium collagenase (ColH) collagen-binding polypeptide, and the therapeutic polypeptide can include interleukin-10 (IL-10) or interleukin-1 receptor antagonist (IL-1Ra). In some examples, the fusion proteins can include a linker polypeptide between the anchor domain and therapeutic polypeptide. Further disclosed herein are isolated nucleic acid molecules encoding these fusion proteins.

Also disclosed herein are methods for treating a subject with a disorder that affects the eye. In some embodiments, the methods include administering to the eye of the subject a therapeutically effective amount of the fusion proteins disclosed herein. In non-limiting examples, the therapeutic polypeptide can include IL-1Ra, IL-10, P17, P144, an inhibitor of TNFα polypeptide from the TNF receptor 1, or an antibody that specifically binds TGFβ, TNFα, IL-1β, IL-6, or IFNγ. In other embodiments, the therapeutic polypeptide is an Ig-binding polypeptide. These methods can also include administering to the subject a therapeutically effective amount of an antibody.

Disclosed herein are also pharmaceutical compositions that include a therapeutically effective amount of the fusion proteins disclosed herein and a pharmaceutically acceptable carrier formulated for external and/or topical administration to the eye. Further disclosed herein are unit dose dispensers for dispensing the pharmaceutical compositions.

### BRIEF DESCRIPTION OF THE FIGURES

FIGS. 1A-1B.: Anchor domains can be used to attach heterologous proteins rapidly to surfaces of eye. FIG. 1A: Binding of fusion proteins to collagen columns. FIG. 1B: Binding of fusion proteins to wounded rabbit eyes.

FIGS. 2A-2B: Binding of lectins to eyes with lesions. FIG. 2A: The results of lectin binding in two experiments are shown. FIG. 2B: The results for wheat germ agglutinin (WGA) binding with and without N-Acetylglucosamine (GlnAc;i.e., the receptor sugar for WGA) is shown.

FIGS. 3A-3B. Schematic of exemplary fusion proteins for treating eye diseases. FIG. 3A shows some embodiments of fusion proteins for treating corneal haze and scarring. FIG. 3B shows some embodiments of fusion proteins for treating dry eye disease (DED) and inflammation.

FIGS. 4A-4B: Binding of fusion proteins to target molecules. FIG. 4A shows that a fusion protein with a heparin-binding (HS) polypeptide retains heparin-binding activity. FIG. 4B shows that a fusion protein with a collagen-binding polypeptide from von Willebrand factor (vWF) retains collagen-binding activity.

FIGS. 5A-5B: FIG. 5A shows binding of WGA conjugated polyclonal antibodies to GlnAc agarose. FIG. 5B shows binding of WGA conjugated therapeutic antibodies to GlnAc agarose.

FIGS. 6A-6B: FIG. 6A shows that wounded rabbit eyes bind fusion proteins that include P17 and P144. FIG. 6B shows that wounded rabbit eye binds WGA-conjugated antibodies.

FIGS. 7A-7B: FIG. 7A shows binding of labeled IL-1β to a WGA-conjugated antibody. FIG. 7B shows that an adapter (Ig-binding polypeptide) mediates binding of antibodies to collagen.

FIG. 8: The time course of bound WGA showing that it remains for prolonged time at the surface of the eye.

FIG. 9: The lectins jacalin and concanavalin A (conA) show binding activity when covalently bound to antibodies.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on September 19, 2017, 18.4 KB, which is incorporated by reference herein. In the accompanying sequence listing:

SEQ ID NO: 1 is an amino acid sequence of a collagen-binding anchor domain from the von Willebrand factor (vWF).

SEQ ID NO: 2 is an amino acid sequence of a collagen-binding anchor domain from the von Willebrand factor (vWF).

SEQ ID NO: 3 is an amino acid sequence of a collagen-binding anchor domain from Clostridium collagenase (ColH).

SEQ ID NO: 4 is an amino acid sequence of a heparin-binding (HS) anchor domain.

SEQ ID NO: 5 is an amino acid sequence of a heparin-binding (HS) anchor domain.

SEQ ID NO: 6 is an amino acid sequence of a carbohydrate-binding anchor domain from concanavalin A (ConA).

SEQ ID NO: 7 is an amino acid sequence of a carbohydrate-binding anchor domain from wheat germ agglutinin (WGA).

SEQ ID NO: 8 is an amino acid sequence of a carbohydrate-binding anchor domain from jacalin (Jac).

SEQ ID NO: 9 is an amino acid sequence of a linker.

SEQ ID NO: 10 is an amino acid sequence of a protein A immunoglobulin (Ig)-binding fragment.

SEQ ID NO: 11 is an amino acid sequence of a protein G immunoglobulin (Ig)-binding fragment.

SEQ ID NO: 12 is an amino acid sequence of an inhibitor of TNFα polypeptide from the TNF receptor 1.

SEQ ID NO: 13 is an amino acid sequence of a P144 polypeptide.

SEQ ID NO: 14 is an amino acid sequence of a P17 polypeptide.

SEQ ID NO: 15 is an amino acid sequence of a fusion peptide that includes a protein G polypeptide as the Ig-binding polypeptide and a vWF polypeptide as an anchor domain.

SEQ ID NO: 16 is an amino acid sequence of a fusion peptide that includes a protein G polypeptide as the Ig-binding polypeptide and a vWF polypeptide as an anchor domain.

SEQ ID NO: 17 is an amino acid sequence that includes two copies of P144 as a therapeutic polypeptide and a vWF polypeptide as an anchor domain.

SEQ ID NO: 18 is an amino acid sequence that includes two copies of P17 as a therapeutic polypeptide and a vWF polypeptide as an anchor domain. SEQ ID NO: 19 is an amino acid sequence of a linker.

### DETAILED DESCRIPTION OF SEVERAL EMBODIMENTS

Advances in drug development increasingly depend on understanding and targeting disease mechanisms, and numerous protein-based drugs, such as monoclonal antibodies, molecular traps, and cytokines are currently used clinically (Leader, B. et al., Nat Rev Drug Discov, 7:21-39, 2008; Kling, J., Nat Biotechnol, 32:121-124, 2014; Andrews, L. et al., Hum Exp Toxicol, 34:1279-1285, 2015). Although these agents are used to treat many diseases, such as auto-immune disorders, infections, and inappropriate vascularization, the rapid clearance by the tear flow and blinking has precluded their use to treat ocular surface diseases. The fusion proteins disclosed herein allow for retention of therapeutic proteins in the eye, specifically at the ocular surface, for longer periods of time.

The compositions and methods herein provide local drug delivery to the eye; do not require a delivery device, as they allow local drug delivery in the form of standard eye drops (but can be used with a delivery device); and do not interfere with vision. These features enhance patient acceptance and compliance and are important improvements over current delivery systems and will extend the arsenal of therapeutics available to ophthalmologists to use modern biological reagents. The compositions and methods herein circumvent systemic drug delivery and permit using drugs that would otherwise have unacceptable side effects.

The regions on the ocular surface that are targeted can be regulated precisely using anchor domains. Disease processes commonly disrupt epithelial coverage, and anchor domains that have an affinity to collagen will specifically bind such sites. Anchors that bind heparan sulfate are expected to concentrate at regions denuded of epithelium but will also bind the outer surface of the corneal epithelium and conjunctiva. Appropriate lectins also can be used to bind to the glycocalyx of epithelia and target the entire ocular surface.

Disclosed herein are fusion proteins that include an anchor domain and a therapeutic polypeptide, which can be used to treat a disorder that affects the eye, such as, but not limited to, corneal haze and scarring, dry eye disease, and ocular inflammation. In some embodiments, the fusion proteins can include an anchor domain, such as a lectin carbohydrate-binding polypeptide, a von Willebrand factor (vWF) collagen-binding polypeptide, a Clostridium collagenase (ColH) collagen-binding polypeptide, or a heparin-binding (HS) polypeptide, and the therapeutic polypeptide can include a transforming growth factor beta 1 (TGFβ) antagonist, a tumor necrosis factor alpha (TNFα) antagonist, an interleukin (IL)-1β antagonist, an IL-6 antagonist, an immunoglobulin-binding (Ig) polypeptide, or an interferon (IFN)γ antagonist. In other embodiments, the fusion proteins can include an anchor domain, such as a lectin carbohydrate-binding polypeptide, a von Willebrand factor (vWF) collagen-binding polypeptide, or a Clostridium collagenase (ColH) collagen-binding polypeptide, and the therapeutic polypeptide can include interleukin-10 (IL-10) or interleukin-1 receptor antagonist (IL-1Ra). In some examples, the fusion proteins can include a linker polypeptide between the anchor domain and therapeutic polypeptide. Further disclosed herein are isolated nucleic acid molecules encoding these fusion proteins.

In some non-limiting examples, the fusion proteins can include a lectin carbohydrate-binding polypeptide from wheat germ agglutinin (WGA), jacalin (Jac), or concanavalin A (conA). In other non-limiting examples, the TGFβ antagonist can include P17, P144, or an antibody that specifically binds TGFβ; the TNFα antagonist can include an inhibitor of TNFα polypeptide from the TNF receptor 1 or an antibody that specifically binds TNFα; the IL-1β antagonist can include an antibody that specifically binds IL-1β; the IL-6 antagonist can include an antibody that specifically binds IL-6; the IFNγ antagonist can include an antibody that specifically binds IFNγ; and/or the Ig-binding polypeptide can include protein A, protein G, or protein L or an Ig-binding fragment thereof.

In some embodiments, the fusion proteins herein can include a lectin carbohydrate-binding protein as the anchor domain and the inhibitor of TNFα polypeptide from the TNF receptor 1, IL-10, IL-1Ra, or an antibody that specifically binds TGFβ, TNFα, IL-1β, IL-6, or IFNγ as the therapeutic polypeptide. In other embodiments, the fusions proteins can include a vWF collagen-binding polypeptide or ColH collagen-binding polypeptide as the anchor domain and the inhibitor of TNFα polypeptide from the TNF receptor 1, IL-1Ra, IL-10, P17, P144, or an Ig-binding polypeptide as the therapeutic polypeptide. In still further embodiments, the fusion proteins can include an HS polypeptide as the anchor domain and an inhibitor of TNFα polypeptide from the TNF receptor 1 as the therapeutic polypeptide. In some examples, the lectin carbohydrate-binding polypeptide includes the amino acid sequence of SEQ ID NO: 6, SEQ ID NO: 7, or SEQ ID NO: 8; the vWF collagen-binding polypeptide includes the amino acid sequence of SEQ ID NO: 1; the ColH collagen-binding polypeptide includes the amino acid sequence of SEQ ID NO: 3; and/or the HS polypeptide includes the amino acid sequence of SEQ ID NO: 5.

In some examples, the fusion proteins are of use in treating corneal haze or corneal scarring. In some embodiments, the fusion protein can include a vWF collagen-binding polypeptide as the anchor domain and P17, P144, or the Ig-binding polypeptide as the therapeutic polypeptide. In other embodiments, the fusion proteins can include a lectin carbohydrate-binding polypeptide as the anchor domain and an antibody that specifically binds TGF(3 as the therapeutic polypeptide.

In other examples, fusion proteins are of use in treating dry eye or inflammation at an ocular surface. In some embodiments, the fusion proteins include a vWF collagen-binding polypeptide as the anchor domain and an Ig-binding polypeptide, the inhibitor of TNFα polypeptide from the TNF receptor 1, IL-1Ra, or IL-10 as the therapeutic polypeptide. In other embodiments, the fusion proteins include a lectin carbohydrate-binding polypeptide as the anchor domain and the inhibitor of TNFα polypeptide from the TNF receptor 1, IL-1Ra, IL-10, or an antibody that specifically binds TNFα, IL-1β, IL-6, or IFNγ as the therapeutic antibody. In still further embodiments, the fusion proteins can include, an HS polypeptide as the anchor domain and an inhibitor of TNFα polypeptide from the TNF receptor 1 as the therapeutic polypeptide.

In further examples, the fusion protein includes a therapeutic proteins that it an Ig-binding polypeptide, such as protein A, protein G, or protein L. In these embodiments, methods of treatment also include administering a therapeutically effective amount of an antibody that specifically binds a polypeptide of interest.

Further disclosed herein is an isolated nucleic acid molecule encoding the fusion proteins.

In some examples, the isolated nucleic acid molecule is operably linked to a heterologous promoter. In still further examples, the isolated nucleic acid molecule can be included in an expression vector.

Also disclosed herein are methods for treating a subject with a disorder that affects the eye. In some examples, the methods can include administering to the eye of the subject a therapeutically effective amount of the fusion proteins disclosed herein. In some embodiments, the fusion proteins can include IL-1Ra, IL-10, P17, P144, the inhibitor of TNFα polypeptide from the TNF receptor 1, or the antibody that specifically binds TGFβ, TNFα, IL-1β, IL-6, or IFNγ as therapeutic polypeptide. In other embodiments, the methods can include administering to the eye of the subject a therapeutically effective amount of the fusion proteins with an Ig-binding polypeptide as the therapeutic polypeptide and any therapeutically effective antibody.

In some exemplary methods, the subject has corneal haze or corneal scarring. In some non-limiting embodiments, the methods include administering to the eye of the subject a therapeutically effective amount of the fusion proteins disclosed herein with P17, P144, or an antibody that specifically binds TGFβ as the therapeutic polypeptide. In other non-limiting embodiments, the methods include administering to the eye of the subject a therapeutically effective amount of the fusion proteins disclosed herein with an Ig-binding polypeptide as the therapeutic polypeptide and an antibody that specifically binds TGFβ.

In other exemplary methods, the subject has dry eye or inflammation of an ocular surface. In some non-limiting embodiments, the methods can include administering to the eye of the subject a therapeutically effective amount of the fusion proteins disclosed herein with IL-1Ra, IL-10, an inhibitor of TNFα polypeptide from TNF receptor 1, or an antibody that specifically binds TNFα, IL-1β, IL-6, or IFNγ as the therapeutic polypeptide. In other non-limiting embodiments, the methods include administering to the eye of the subject a therapeutically effective amount of the fusion proteins disclosed herein with an Ig-binding polypeptide as the therapeutic polypeptide and an antibody that specifically binds TNFα, IL-1β, IL-6, or IFNγ. In some examples, the subject has Sjögren's syndrome. In other examples, the subject has inflammation caused by an acanthamoebal, a bacterial, a fungal, helminth, toxoplasma, or a viral infection. In some non-limiting examples, the subject has keratitis caused by a bacterial infection. In other non-limiting examples, the subject has inflammation caused by glaucoma, uveitis, macular degeneration, diabetic retinopathy, or other retinal problem.

Disclosed herein are pharmaceutical compositions that include a therapeutically effective amount of the fusion proteins disclosed herein and a pharmaceutically acceptable carrier formulated for external and/or topical administration to the eye. Further disclosed herein are unit dose dispensers for dispensing the pharmaceutical compositions.

In some examples, the pharmaceutical compositions include a lectin carbohydrate-binding protein as the anchor domain and the inhibitor of TNFα polypeptide from the TNF receptor 1, IL-1Ra, IL-10, or the antibody that specifically binds TGFβ, TNFα, IL-1β, IL-6, or IFNγ as the therapeutic polypeptide. In other examples, the pharmaceutical compositions include a vWF collagen-binding polypeptide as the anchor domain and the inhibitor of TNFα polypeptide from the TNF receptor 1, IL-1Ra, IL-10, P17, or P144 as the therapeutic polypeptide. In still further examples, the pharmaceutical compositions include an HS polypeptide as the anchor domain and an inhibitor of TNFα polypeptide from the TNF receptor 1 as the therapeutic polypeptide. In certain examples, the pharmaceutical compositions include a fusion protein with the amino acid sequence of SEQ ID NO: 17 or SEQ ID NO: 18.

In some embodiments, the pharmaceutical compositions include an Ig-binding polypeptide as the therapeutic protein. In some embodiments where the pharmaceutical compositions include an Ig-binding polypeptide, the pharmaceutical compositions also include a therapeutically effective antibody. In some non-limiting examples, the pharmaceutical compositions include an antibody that specifically binds TGFβ, TNFα, IL-1β, IL-6, or IFNγ as the therapeutically effective antibody. In other non-limiting examples, the anchor domain includes a vWF collagen-binding polypeptide. In certain examples, the pharmaceutical compositions include SEQ ID NO: 15 or SEQ ID NO: 16.

### I. TERMS

Unless otherwise noted, technical terms are used according to conventional usage. Definitions of common terms in molecular biology can be found in Lewin B, Genes VII, 1999; Kendrew et al., The Encyclopedia of Molecular Biology, 1994; Meyers R, Molecular Biology and Biotechnology: a Comprehensive Desk Reference, 1995; and other similar references.

As used herein, the singular forms “a,” “an,” and “the” refer to both the singular as well as plural unless the context clearly indicates otherwise.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments, the following explanations of terms are provided.

Acanthamoeba keratitis: A rare but serious infection of the eye that can result in permanent visual impairment or blindness. This infection is caused by a microscopic, free-living ameba (single-celled living organism) called Acanthamoeba.

Administration/delivery: To provide or give a subject an agent by any effective route. Exemplary routes of administration include, but are not limited to, topical application, such as eye drops. For example, if the chosen route is local, the composition is administered by introducing the composition into the eye, such as onto the ocular surface of the subject. The term also encompasses long-term administration, such as is accomplished using a continuous release formulation.

The three primary methods of delivery of ocular medications to the eye are topical, local ocular, and systemic. The most appropriate method of administration depends on the area of the eye to be medicated. The conjunctiva, cornea, anterior chamber, and iris typically respond well to topical therapy. Typically, the eyelids can be treated with topical therapy but more frequently require systemic therapy.

Amino acid substitution: The replacement of one amino acid in peptide with a different amino acid.

Agent: Any substance or any combination of substances that is useful for achieving an end or result. Agents include proteins, nucleic acid molecules, compounds, small molecules, organic compounds, inorganic compounds, or other molecules of interest, such as viruses, such as recombinant viruses. An agent can include a therapeutic agent, a diagnostic agent, or a pharmaceutical agent. A therapeutic agent is a substance that demonstrates some therapeutic effect by restoring or maintaining health, such as by alleviating the symptoms associated with a disease or physiological disorder, delaying (including preventing) progression or onset of a disease, or allowing for a specific molecule with a biological function to be retained locally. In some embodiments, the agent is a polypeptide agent. For example, Protein A, Protein G, and Protein L are therapeutic agents because they can bind a therapeutic antibody and thus achieve therapeutic efficacy when used with a monoclonal or polyclonal antibody. The skilled artisan will understand that particular agents may be useful to achieve more than one result.

Anchor domain: A protein domain that can attach to cells or a tissue or by binding extracellular molecules, such as collagen, heparan sulfate, or the glycocalyx. Anchor domains generally bind molecules at the ocular surface through non-covalent bonds.

Animal: Living multi-cellular vertebrate organisms, a category that includes, for example, mammals and birds. The term mammal includes both human and non-human mammals. Similarly, the term “subject” includes both human and veterinary subjects.

Antibody: A polypeptide substantially encoded by an immunoglobulin gene or immunoglobulin genes or fragments thereof, which specifically binds and recognizes an analyte (antigen) Immunoglobulin genes include the kappa, lambda, alpha, gamma, delta, epsilon, and mu constant region genes as well as myriad immunoglobulin variable region genes.

Antibodies exist, for example, as intact immunoglobulins and as a number of well-characterized fragments produced by digestion with various peptidases, such as Fabs, Fvs, and single-chain Fvs (SCFvs). Included are intact immunoglobulins as well as the variants and portions thereof that are well known in the art, such as Fab′ fragments, F(ab)₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while, in dsFvs, the chains have been mutated to introduce a disulfide bond to stabilize the association of the chains. The term also includes genetically engineered forms, such as chimeric antibodies (e.g., humanized murine antibodies) and heteroconjugate antibodies (e.g., bispecific antibodies). See also, Pierce Catalog and Handbook, 1994-1995 (Pierce Chemical Co., Rockford, IL); Kuby, J., Immunology, 3 rd Ed., W. H. Freeman & Co., New York, 1997.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies. In some examples, monoclonal antibodies are isolated from a subject. The amino acid sequences of such isolated monoclonal antibodies can be determined.

A “polyclonal antibody” is an antibody that is secreted by different B cell lineages within the body (whereas monoclonal antibodies come from a single cell lineage). It includes a collection of immunoglobulin molecules that react against a specific antigen, each identifying a different epitope.

Antigen: A polypeptide that can stimulate the production of antibodies or a T cell response in an animal, including polypeptides that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Autoimmune disorder: Autoimmune diseases are a broad range of related diseases in which a person's immune system produces an inappropriate response against its own cells, tissues and/or organs, resulting in inflammation and damage. There are over 80 different autoimmune diseases. Some autoimmune diseases affect mainly one part of the body (such as multiple sclerosis, autoimmune thyroid disease, and type 1 diabetes), while others can affect many parts of the body (such as systemic lupus erythematosus, rheumatoid arthritis, systemic vasculitis, and Sjögren's syndrome).

Bacterial keratitis: Keratitis caused by a bacterial infection of the cornea (i.e., the clear, round dome covering the eye's iris and pupil) that causes pain, reduced vision, light sensitivity, and tearing or discharge from your eye. Bacterial keratitis often results from infection caused by contact lens use or injury to the eye, usually develops very quickly and, if left untreated, can cause blindness. Superficial keratitis involves the uppermost layers of the cornea. When this form of keratitis has healed, there is usually no scar on the cornea. Deep keratitis affects deeper corneal layers. There can be a scar left after healing which may or may not affect vision, depending on where the scar is located. Keratitis can also be caused by acanthamoeba, fungal, and viral infections.

Chronic condition: A human health condition or disease that is persistent or otherwise long-lasting in its effects or a disease that comes with time. The term chronic is often applied when the course of the disease lasts for more than three months. Common chronic diseases include autoimmune diseases such as Sjögren's syndrome.

Clostridium collagenase (ColH): One of several bacterial collagenases that have been widely studied for their biochemical and enzymatic properties. It is a metalloproteinase that is able to digest triple-helical type I, II, and III collagen into simple peptides under physiological conditions. Examples of sequences are available in GENBANK® (e.g., Accession Nos. D29981.1 and BAA06251.1, incorporated by reference herein as available on Sep. 14, 2017, which provide exemplary nucleotide and protein sequences for ColH); other examples are available herein (e.g., SEQ ID NO: 3).

Collagen: The main structural protein in the extracellular space in the various connective tissues in animal bodies. As the main component of connective tissue, it is the most abundant protein in mammals, making up from 25% to 35% of the whole-body protein content. Depending upon the degree of mineralization, collagen tissues may be rigid (bone), compliant (tendon), or have a gradient from rigid to compliant (cartilage). Collagen, in the form of elongated fibrils, is mostly found in fibrous tissues, such as tendons, ligaments, and skin. It is also abundant in corneas, cartilage, bones, blood vessels, the gut, intervertebral discs, and the dentin in teeth. In muscle tissue, it serves as a major component of the endomysium. Collagen constitutes one to two percent of muscle tissue and accounts for 6% of the weight of strong, tendinous muscles. The fibroblast is the most common cell that creates collagen. Twenty-eight types of collagen have been identified. Collagen includes fibrillar (Types I, II, II, V, XI) and non-fibrillar collagen.

Concanavalin A (ConA): A lectin (carbohydrate-binding protein) from the jack-bean, Canavalia ensiformis. It binds to mainly internal and non-reducing terminal α-D-mannosyl and α-D-glucosyl groups. ConA binds to surfaces of many cell types and is widely used in biology and biochemistry to characterize glycoproteins and other sugar-containing molecules on the surface of cells. It is also used to purify glycoproteins by affinity chromatography. ConA sequences are publicly available. GENBANK® Accession Nos. AAL09432.1 and AF308777.1, incorporated herein by reference as available on Sep. 14, 2017, provide exemplary jack bean ConA protein and nucleotide sequences, respectively. Other examples are available herein (e.g., SEQ ID NO: 6).

Contacting: Placement in direct physical association; includes both in solid and liquid form. Contacting includes contact between one molecule and another molecule, for example the amino acid on the surface of one polypeptide, such as a therapeutic peptide, that contacts another polypeptide. Contacting can also include contacting a cell for example by placing a peptide in direct physical association with a cell.

Control: A reference standard. In some embodiments, the control is a sample obtained from a healthy patient. In other embodiments, the control is a tissue sample obtained from a patient diagnosed with an eye disorder. In still other embodiments, the control is a historical control or standard reference value or range of values (e.g., a previously tested control sample with a known prognosis or outcome or group of samples that represent baseline or normal values). A difference between the value of a parameter measured in a test sample and a control can be an increase or a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Conjunctiva: A thin, clear, moist membrane that coats the inner surfaces of the eyelids (i.e., palpebral conjunctiva) and the outer surface of the eye (i.e., ocular, or bulbar, conjunctiva). The conjunctiva lines the inside of the eyelids and covers the sclera (i.e., the white of the eye); it is composed of a non-keratinized, stratified columnar epithelium with goblet cells and a stratified columnar epithelium. The conjunctiva is highly vascularized with many microvessels that are easily accessible for imaging studies. The conjunctiva helps lubricate the eye by producing mucus and tears, although at a smaller volume of tears than the lacrimal gland. It also contributes to immune surveillance and helps prevent microbes from entering the eye.

Corneal diseases: A variety of conditions that affect mainly the cornea, including infections, degenerations, and many other disorders of the cornea that may arise mostly as a result of heredity. The cornea is the clear, protective outer layer of the eye. It serves as a barrier against dirt, germs, and other particles that can harm the eye's delicate components. The “cornea” is also capable of filtering out some of the sun's ultraviolet light and plays a key role in vision. As light enters the eye, it is refracted, or bent, by the outside shape of the cornea. The curvature of this outer layer helps determine how well your eye can focus on objects close-up and far away. If the cornea of your eye becomes damaged through disease, infection, or injury, the resulting scars can interfere with vision by blocking or distorting light as it enters the eye.

Corneal haze: A cloudy or opaque appearance of the cornea (see U.S. Pat. No. 6,143,315 and U.S. Patent Pub No. 2003/0153524, both incorporated herein by reference). This part of the eye can become damaged through disease, infection, or injury, and the resulting scarring can interfere with vision by blocking or distorting light as it enters the eye. The cornea is normally clear, so corneal haze can greatly impair vision. Although the haze can occur in any part of the cornea, it is most often found within the thicker, middle layer of the cornea, which is referred to as the stroma. Corneal haze can cause vision to be blurry, unfocused, or obscured. It can also cause halos to be visible around light, especially at night or in the dark. Corneal haze most often appears after suffering a trauma, infection, or surgery. It is usually caused by inflammatory cells that is activated inside the eye. Corneal haze also sometimes occurs during laser vision correction procedures.

Corneal scarring: Scarring caused by injury to the cornea (abrasion, laceration, burns, or infections); depending on the degree of scarring, vision can range from a blur to total blindness (see U.S. Pat. No. 6,143,315 and U.S. Patent Pub No. 2003/0153524, both incorporated herein by reference). Although extremely painful, surface abrasions heal transparently and do not leave scars. Deeper abrasions and ulcerations/lacerations result in a loss of corneal tissue, which is replaced by scar tissue. Scars from burns depend on the type and depth of burn; boiling water or a curling iron leave superficial scarring, and acids or alkalies cause deeper damage unless neutralized immediately. Scarring from disease (usually inflammation) is typically the result of a proliferation of new blood vessels into the clear cornea to assist in the healing process.

Cytokines: A broad category of small proteins (approximately 5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumor necrosis factors but generally not hormones or growth factors. Cytokines are produced by a broad range of cells, including immune cells, such as macrophages, B lymphocytes, T lymphocytes, and mast cells, as well as endothelial cells, fibroblasts, and various stromal cells; a given cytokine may be produced by more than one type of cell. Cytokines are important in health and disease, specifically in host responses to infection, immune responses, inflammation, trauma, sepsis, cancer, and reproduction. They act through receptors and are especially important in the immune system; cytokines modulate the balance between humoral and cell-based immune responses, and they regulate the maturation, growth, and responsiveness of particular cell populations. Cytokines include interleukins.

Defective innervation: When either the sympathetic or parasympathetic sensory innervation of the lacrimal gland or sensory innervation of ocular surface is malfunctioning. Defective innervation of the eye can result from inflammation, trauma, or surgery.

Diabetic retinopathy: When damage occurs to the retina due to diabetes, also known as diabetic eye disease; it can eventually lead to blindness. Diabetic retinopathy is an ocular manifestation of diabetes that affects up to 80 percent of all patients who have had diabetes for 20 years or more. However, at least 90% of new cases could be reduced with proper and vigilant treatment and monitoring of the eyes. The longer a person has diabetes, the higher his or her chances of developing diabetic retinopathy, and it is the leading cause of blindness for people 20 to 64 years old.

Domain: A domain of a protein is a part of a protein that shares common structural, physiochemical, and functional features, such as hydrophobic, polar, globular, helical domains or properties (e.g., a collagen-binding, carbohydrate-binding, or heparan sulfate-binding domain). A domain can also be a functional domain that has a particular enzymatic activity or feature, such as a domain of protein A, protein G, or protein L that binds monoclonal or polyclonal antibodies.

Dry eye: A condition that occurs when either the eye does not produce enough tears or when the tears evaporate too quickly. This can result from meibomian gland dysfunction, allergies, pregnancy, Sjögren's syndrome, vitamin A deficiency, LASIK surgery, certain medications (e.g., antihistamines), certain blood pressure medication, hormone replacement therapy, and antidepressants. Chronic conjunctivitis, such as from tobacco smoke exposure or infection, may also lead to dry eye. Diagnosis is based on symptoms, evaluation of tear secretion, and/or staining of the ocular surface.

Dry eye disease (DED): A condition also known as dry eye syndrome (DES), keratoconjunctivitis sicca (KCS), and keratitis sicca, which is a multifactorial disease of the tears and the ocular surface that results in discomfort, visual disturbance, and tear film instability with potential damage to the ocular surface. Dry eye syndrome is a common form of ocular surface disease (OSD) and may overlap with other causes of OSD, such as ocular allergy and meibomian gland dysfunction (MGD). Dry eye syndrome includes symptoms caused by multiple conditions, such as keratoconjunctivitis sicca, Sjögren's syndrome, corneal injury, age-related dry eye, Stevens-Johnson syndrome, congenital alachrima, side effects from a drug or drugs, infection, Riley-Day syndrome, conjunctival fibrosis, eye stress, glandular and tissue destruction, ocular cicatrical pemphogoid, blepharitis, autoimmune and other immunodeficient disorders, allergies, diabetes, lacrimal gland deficiency, lupus, Parkinson's disease, Sjögren's syndrome, rheumatoid arthritis, rosacea, environmental exposure to excessively dry air, airborne particulates, smoke, smog, and an inability to blink (see U.S. Patent Pub. No. 2006/0281739, incorporated herein by reference). Symptoms of DED include a burning sensation, itchy eyes, aching sensations, heavy eyes, fatigued eyes, sore eyes, a dryness sensation, red eyes, photophobia (light sensitivity), blurred vision, foreign body sensation (i.e., a feeling that grit or some other object or material is in the eye), and watery eyes.

Epithelium: Epithelial tissues line the cavities and surfaces of blood vessels and organs throughout the body. These tissues can be arranged in a single layer of cells as a simple epithelium, either squamous, columnar, or cuboidal, or in layers of two or more cells deep as a stratified (layered) epithelium, either squamous, columnar, or cuboidal. All glands are made up of epithelial cells. Functions of epithelial cells include secretion, selective absorption, protection, transcellular transport, and sensing. Epithelial layers contain no blood vessels; therefore, they must receive nourishment via diffusion of substances from the underlying connective tissue through the basement membrane.

The “corneal epithelium” is made up of epithelial tissue and covers the front of the cornea. It acts as a barrier to protect the cornea, resisting the free flow of fluids from the tears, and prevents bacteria from entering the epithelium and corneal stroma.

Expression: Translation of a nucleic acid into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which they are operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components the presence of which can influence expression and can also include additional components the presence of which is advantageous, such as leader sequences and fusion partner sequences. Expression control sequences can include a promoter.

A promoter is a minimal sequence sufficient to direct transcription. Also included are promoter elements that are sufficient to render promoter-dependent gene expression controllable for cell-type specificity or tissue-specificity or that is inducible by external signals or agents; such elements may be located in the 5′ or 3′ regions of the gene. Both constitutive and inducible promoters are included (see, for example, Bitter et al., Methods in Enzymology 153:516-544, 1987). Promoters produced by recombinant DNA or synthetic techniques may also be used to provide for transcription of the nucleic acid sequences.

Expression vector: A vector that includes a recombinant polynucleotide comprising expression control sequences operatively linked to a nucleotide sequence to be expressed.

An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

Eye disorder: Any disturbance, defect, or abnormality in eye function or structure, such as corneal haze and scarring, dry eye disease, or ocular inflammation. An eye disorder can be congenital, hereditary, or the result of a trauma, such as an injury, an illness, inflammation, an autoimmune disease, an infection (e.g., an acanthamoebal, a viral, a bacterial, or a fungal infection), a foreign body, ultraviolet light exposure, contact lens overwear, or a drug.

Fusion protein: A composite protein made up of two (or more) distinct, heterologous polypeptides, which are not normally covalently bound together. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid or by chemical synthesis methods (i.e., chemical conjugation) that are well-known in the art. A fusion protein can include an anchor domain and a therapeutic polypeptide as well as, optionally, a linker between the anchor domain and the therapeutic polypeptide.

Glaucoma: A group of eye diseases that result in damage to the optic nerve and vision loss. The most common type is open-angle glaucoma, which develops slowly over time without pain. Side vision may begin to decrease followed by central vision resulting in blindness if not treated. The less common form, closed-angle glaucoma can present gradually or suddenly; sudden presentation may involve severe eye pain, blurred vision, mid-dilated pupil, redness of the eye, and nausea. Vision loss from glaucoma, once it has occurred, is permanent.

Glycocalyx: The glycocalyx is a glycoprotein-polysaccharide covering that surrounds the cell membranes of certain bacteria, epithelia, and other cells. Most animal epithelial cells have a fuzz-like coat on the external surface of their plasma membranes. This coating consists of several carbohydrate moieties of membrane glycolipids and glycoproteins, which serve as backbone molecules for support. Generally, the carbohydrate portion of the glycolipids found on the surface of plasma membranes helps these molecules contribute to cell-cell recognition, communication, and intercellular adhesion.

Heparin: A polysacceride present in mast cells which is extensively used as an anticoagulant (Oduah, E. I. et al Pharmaceuticals 9, 38; doi:10.3390, 2016). It is structurally very similar to heparan sulfate and the HS domain bind to both heparin and heparan sulfate with high affinity.

Heparan sulfate (HS): A linear polysaccharide found in all animal tissues, occurring as a proteoglycan (HSPG) in which two or three HS chains are attached in close proximity to cell surface or extracellular matrix proteins. In this form, HS binds a variety of protein ligands and regulates a wide variety of biological activities, including developmental processes, angiogenesis, blood coagulation, abolishing detachment activity by GrB (Granzyme B), and tumor metastasis. Heparan sulfate is also a cellular receptor for a number of viruses. It is structurally very similar to heparin.

Immunoglobulins: A class of proteins found in plasma and other body fluids that exhibit antibody activity and binds with other molecules with a high degree of specificity; immunoglobulins are divided into five classes (IgM, IgG, IgA, IgD, and IgE) on the basis of structure and biological activity Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125, 023; Faoulkner et al., Nature 298:286, 1982; Morrison, J. Immunol. 123:793, 1979; Morrison et al., Ann Rev Immunol 2:239, 1984, all of which are incorporated herein by reference).

Naturally occurring immunoglobulins are made up of four polypeptide chains. There are two long chains referred to as “heavy” or “H” chains, which weigh between about 50 and 75 kilodaltons, and two short chains referred to as “light” or “L” chains, which weigh about 25 kilodaltons. These chains are linked together by disulfide bonds to form a “Y”-shaped molecule. Each heavy chain and light chain can be divided into a variable region and a constant region. An Fc region includes the constant regions of the heavy and the light chains, but not the variable regions. Fc receptors are receptors that specifically bind an Fc region of an immunoglobulin.

Interferon gamma (IFNγ): Also known as IFNG, IFG, and IFN immune (IFI; e.g., OMIM 147570), IFNγ is a cytokine that binds the IFNγ receptor (IFNγR) and exhibits antiviral, antibacterial, and antiprotozan activity. IFNγ is primarily expressed by leukocytes, such as T cells, and aberrant expression of IFNγ has been implicated in various autoinflammatory and autoimmune diseases. Exemplary protein and nucleotide sequences for IFNγ are available at GENBANK® (e.g., Accession Nos. AAB59534.1 and NM_000619.2, respectively, incorporated by reference herein as available on Sep. 14, 2017).

Interleukin 1-beta (IL-1β): Also known as IL1B, IL1-beta, leukocytic pyrogen, leukocytic endogenous mediator, mononuclear cell factor, and lymphocyte activating factor (e.g., OMIM 147720), IL-1β is a cytokine produced by activated macrophages is an important mediator of the inflammatory response. IL-1β is involved in a variety of cellular activities, including cell proliferation, differentiation, and apoptosis. Increased production and/or activity of IL-1β causes multiple autoinflammatory syndromes and has been linked to susceptibility to cancer and tuberculosis. Exemplary protein and nucleotide sequences for IL-1β are available at GENBANK® (e.g., Accession Nos. NP_000567.1 and NM_000576.2, respectively, incorporated by reference herein as available on Sep. 14, 2017).

Interleukin (IL)-1 receptor antagonist (Ra): A member of the IL-1 cytokine family IL-1Ra is secreted by various types of cells, including immune cells, epithelial cells, and adipocytes, and is a natural inhibitor of the pro-inflammatory effect of IL1β. This protein inhibits the activities of interleukin 1, alpha (ILIA) and interleukin 1, beta (IL1B) and modulates a variety of interleukin 1-related immune and inflammatory responses. Exemplary protein and nucleotide sequences for IL-1Ra are available at GENBANK® (e.g., Accession Nos. CAA36262.1 and AJ005835.1, respectively, incorporated by reference herein as available on Sep. 18, 2017).

Interleukin 6 (IL-6): Also known as interferon beta-2; IFNB2, B-cell differentiation factor, B-cell stimulatory factor 2 (BSF2), hepatocyte stimulatory factor (HSF), and hybridoma growth factor (HGF; e.g., OMIM 147620), IL-6 is an interleukin that acts as both a pro-inflammatory cytokine and an anti-inflammatory myokine. IL-6 is secreted by T cells and macrophages to stimulate immune response (e.g., during infection and after trauma, especially burns or other tissue damage leading to inflammation) and has been implicated in the inflammatory and autoimmune processes of various diseases. Exemplary protein and nucleotide sequences for IL-6 are available at GENBANK® (e.g., Accession Nos. P05231.1 and NM_001318095.1, respectively, incorporated by reference herein as available on Sep. 14, 2017).

Interleukin 10 (IL-10): Also known as human cytokine synthesis inhibitory factor (CSIF; e.g., OMIM 124092), IL-10 is an anti-inflammatory cytokine. IL-10 signals through a receptor complex consisting of two IL-10 receptor-1 and two IL-10 receptor 2 proteins. Multiple disease states, such as inflammatory diseases, are associated with low levels of IL-10, but IL-10 has mostly been shown to have immunosuppressive but can also have immunostimulatory effects, depending on the physiological context. Examples of sequences are available in GENBANK® (e.g., Accession Nos. AAI04253.1 and NM_000572.2, incorporated by reference herein as available on Sep. 14, 2017, which provide exemplary protein and nucleotide sequences for WGA, respectively). One of ordinary skill in the art can identify additional IL-10 nucleic acid and protein sequences, including IL-10 variants that retain IL-10 biological activity (such as anti-inflammatory activity).

Inflammation: The complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and to initiate tissue repair.

The classical signs of acute inflammation are calor, dolor, rubor, tumor (heat, pain, redness and swelling) and loss of function. Inflammation is a generic response, and therefore it is considered a mechanism of innate immunity, in contrast to adaptive immunity, which is specific for each pathogen. Prolonged inflammation, known as “chronic inflammation,” leads to a progressive shift in the type of cells present at the site of inflammation, such as mononuclear cells, and is characterized by simultaneous destruction and healing of the tissue from the inflammatory process.

“Ocular inflammation” is inflammation of the eye. Chronic inflammation of the eye can lead to a loss of ocular surface integrity and be further complicated by sight-threatening consequences, such as keratinization, scarring, ulceration, and infection. Eye inflammation can be caused by a number of conditions, including infection (e.g., bacterial, fungal, viral, or parasitic infection, such as blepharitis, chalazion, conjunctivitis, dacryocystitis, iritis, keratitis, periorbital cellulitis, scleritis, sinusitis, and stye or bordeolum), allergy (e.g., drug allergies, food allergies, hay fever or allergic reaction to an allergen, and insect bite allergy, including chronic or acute allergies), autoimmune disorders (e.g., ankylosing spondylitis, Behcet's syndrome, dermatomyositis, Graves' disease, juvenile rheumatoid arthritis, multiple sclerosis, psoriatic arthritis, Reiter's syndrome, rheumatoid arthritis, Sjögren's syndrome, systemic lupus erythematosus, and Wegener's granulatomatosis), graft versus host disease, dry eye syndrome, limbal stem cell insufficiency, irritation, and injury or trauma to the eye or eyelid, such as due to blunt trauma, corneal abrasion or ulcer, foreign objects or materials, hematoma, insect bite or sting, irritants, and orbital bone fracture (see U.S. Pat. Pub. No. 2013/0336557, incorporated herein by reference). Symptoms of inflammation in the eye can occur in one or both eyes and span from minutes to years depending on the type and severity of the underlying condition. Symptoms of eye inflammation include itching, excessive tear production, eye discharge, pain, redness, swelling, tearing, and/or unusual warmth or heat (see U.S. Pat. Pub. No. 2013/0336557, incorporated herein by reference).

Inhibiting or treating a disease: Inhibiting the full development of a disease or condition, for example, in a subject who is at risk for a disease, such as corneal haze or scarring, dry eye disease, or ocular inflammation. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated, produced apart from, or purified away from other biological components in the cell of the organism in which the component naturally occurs, such as, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides, and proteins prepared by recombinant expression in a host cell as well as chemically synthesized nucleic acids.

Jacalin (Jac): A lectin from jackfruit seeds that is both a potent T cell mitogen and an apparently T cell-independent activator of human B cells for the secretion of immunoglobulins. Jacalin is a D-Gal-binding lectin and belongs to a family of galactose-binding lectins containing the Jacalin-like lectin domain. The lectin is blood group non-specific after neuraminidase treatment and agglutinates human erythrocytes. Post-translational proteolytic modification of Jacalin yields a novel carbohydrate-binding site involving the N terminus of the a-chain. Jac sequences are publicly available. GENBANK® Accession Nos. AAA32680.1 and L03798.1, incorporated herein by reference as available on Sep. 14, 2017, provide exemplary jackfruit Jac protein and nucleotide sequences, respectively.

Lectins: Lectins are carbohydrate-binding proteins, macromolecules that are highly specific for sugar moieties and occur ubiquitously in nature. Most lectins do not possess enzymatic activity but may bind to a soluble carbohydrate or to a carbohydrate moiety that is a part of a glycoprotein or glycolipid; they typically agglutinate certain animal cells and/or precipitate glycoconjugates. Exemplary lectins include wheat germ agglutinin (WGA), concanavalin A (conA), and jacalin (Jac).

Keratinization: The process in which the cytoplasm of the outermost cells of the mammalian epidermis is replaced by keratin.

Keratitis: An inflammation of the cornea.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Such polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction.

Further, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Ocular surface: The surface of the eye, including the cornea, the conjunctiva, and the tear ducts which connect to them as well as the eyelids.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, e.g., Remington's Pharmaceutical Sciences, 1289-1329, 1990, incorporated herein by reference). Except insofar as any conventional carrier is incompatible with the active ingredient, its use in the therapeutic or pharmaceutical compositions is contemplated.

In general, the nature of the carrier will depend on the particular mode of administration being employed. For example, administration can be local. Local modes of administration include topical application, such as eye drops, or any other pharmaceutical dosage form formulated for administration to the eye. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound, biologic, or composition capable of inducing a desired therapeutic or prophylactic effect when properly administered to a subject or a cell.

Polypeptide: Three or more covalently attached amino acids. The term encompasses proteins, protein fragments, and protein domains. A “collagen-binding” polypeptide is a polypeptide with the ability to specifically bind collagen.

The term “polypeptide” is specifically intended to cover naturally occurring proteins, as well as those which are recombinantly or synthetically produced. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell.

Conservative amino acid substitutions are those substitutions that do not substantially affect or decrease a function of a protein, such as the ability of the protein to bind its target and/or exert a therapeutic effect when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are well known. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that reduce an activity or function of the protein, such as the ability to induce a synergistic response when administered to a subject. For instance, if an amino acid residue is essential for a function of the protein, even an otherwise conservative substitution may disrupt that activity. Thus, a conservative substitution does not alter the basic function of a protein of interest.

Protein A refers to any form of protein A polypeptide or variation thereof that retains immunoglobulin-binding activity. For example, protein A may be a 42 kDa surface protein originally found in the cell wall of the bacteria Staphylococcus aureus and/or a recombinant protein that includes protein A or a variant thereof. Protein A of Staphylococcus aureus is encoded by the spa gene, and it is composed of five homologous Ig-binding domains that fold into a three-helix bundle, each domain of which can bind proteins from many mammalian species, most notably immunoglobulin G. Protein A is used in biochemical research because it can bind immunoglobulins; protein A binds the heavy chain within the Fc region of most immunoglobulins and within the Fab region of the human VH3 family Examples of sequences are available in GENBANK® (e.g., Accession Nos. X61307.1 and AAB05743.1, incorporated by reference as available on Sep. 14, 2017, which provide exemplary nucleotide and protein sequences for protein A); other examples are available herein (e.g., SEQ ID NO: 9).

Protein G refers to any form of protein G polypeptide or variation thereof that retains immunoglobulin-binding activity. For example, protein G may be an immunoglobulin-binding protein expressed in group C and G Streptococcal bacteria, which, similar to protein A, has multiple homologous Ig-binding domains but with differing binding specificities and/or a recombinant protein that includes protein A or a variant thereof. Protein G can be a 65 kDa (G148 protein G) and a 58 kDa (C40 protein G) cell surface protein that can be used to purify antibodies through its binding to the Fab and Fc regions. Examples of sequences are available in GENBANK® (e.g., accession nos. Y00428.1 and CAA37410.1, incorporated by reference as available on Sep. 14, 2017, which provide exemplary nucleotide and protein sequences for protein A); other examples are available herein (e.g., SEQ ID NO: 10).

Protein L refers to any form of protein L polypeptide or variation thereof that retains immunoglobulin-binding activity. For example, protein L may be an antibody-binding protein expressed at the surface of approximately 10% of Peptostreptococcus magnus isolates that contains four or five Ig-binding domains that bind kappa light chains and/or a recombinant protein that includes protein A or a variant thereof. Examples of sequences are available in GENBANK® (e.g., accession nos. L04466.1 and AAA67503.1, incorporated by reference as available on Sep. 14, 2017, which provide exemplary nucleotide and protein sequences for protein A).

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides. A purified population of nucleic acids or proteins is greater than about 90%, 91%, 92%, 93%, 94%, 95%, 96%, 97%, 98%, 99% or 100% pure, or free other nucleic acids or proteins, respectively.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is a protein encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in Smith and Waterman, Adv. Appl. Math., 2:482, 1981; Needleman and Wunsch, J. Mol. Biol., 48:443, 1970; Pearson and Lipman, Proc. Natl. Acad. Sci. USA, 85:2444, 1988; Higgins and Sharp, Gene, 73:237, 1988; Higgins and Sharp, CABIOS, 5:151, 1989; Corpet et al., Nucleic Acids Research, 16:10881, 1988; and Pearson and Lipman, Proc. Natl. Acad. Sci. USA, 85:2444, 1988. Altschul, et al., Nature Genet., 6:119, 1994 presents a detailed consideration of sequence alignment methods and homology calculations.

The NCBI Basic Local Alignment Search Tool (BLAST) (Altschul, et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, sequence identity counted over the full length alignment with the amino acid sequence of the factor using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Sjögren's syndrome: A systemic autoimmune disease in which immune cells attack and destroy the exocrine glands that produce saliva and tears. The hallmark symptoms of the disorder are Xerostomia (dry mouth) and xerophthalmia (conjunctivitis sicca, dry eyes). Sjögren's syndrome can also cause skin, nose and vaginal dryness, and can affect other organs of the body including the kidneys, blood vessels, lungs, liver, pancreas and brain. Sjögren's syndrome can occur in all age groups of both women and men. However, nine out of ten Sjögren's patients are women, with the average age of onset being in the late 40s. Sjögren's syndrome can occur independently, referred to as primary Sjögren's syndrome, or may develop years after the onset of an associated rheumatic disorder, referred to as secondary Sjögren's syndrome.

Xerostomia and xerophthalmia are usually the first detected symptoms of Sjögren's syndrome (Fox et al., Lancet 1, 1432-1435, 1985). It has been postulated that immunologically-activated or apoptotic glandular epithelial cells that expose autoantigens in predisposed individuals could drive autoimmune-mediated tissue injury (see, e.g., Voulgarelis et al., Nat Rev Rheumatol 6, 529-537, 2010; Xanthou et al., Clin Exp Immunol 118, 154-163, 1999) Immune activation in this patient can present as focal, mononuclear (T, B and macrophage) cell infiltrates proximal to the ductal epithelial cells (epithelitis) and forms sialadenitis. CD4+T-lymphocytes constitute 60-70 percent of the mononuclear cells infiltrating the salivary glands (Skopouli et al., J Rheumatol 18, 210-214, 1991). Abnormal activation of proinflammatory Th1 (Bombardierei et al., Arthritis Res Ther 6, R447-R456, 2004; Vosters et al., Arthritis Rheum 60, 3633-3641, 2009) and Th17 (see, e.g., Nguyen et al., Arthritis and Rheumatism 58, 734-743, 2008) cells have been reported in the human or condition and in animal models.

Small molecular weight compounds (small molecules): A low-molecular-weight (<900 daltons) organic compound that may help regulate a biological process. The upper molecular-weight limit for a small molecule is approximately 900 daltons, which allows for the possibility to rapidly diffuse across cell membranes so that they can reach intracellular sites of action. In addition, this molecular weight cutoff is a necessary but insufficient condition for oral bioavailability. This term can also include a molecule that binds to a specific biological target, such as a specific protein or nucleic acid, and acts as an effector, altering the activity or function of the target. Small molecules can have a variety of biological functions, serving as cell signaling molecules, drugs in medicine, pesticides in farming, and in many other roles.

Specifically binding agent: An agent (such as a protein or polypeptide) that binds substantially or preferentially only to a defined target, such as a protein (e.g., TGFβ, TNFα, IL-1β, IL-6, IL-1R, or IFNγ), enzyme, polysaccharide, nucleic acid, or a small molecule. For example, an agent that specifically binds a protein binds substantially only the defined protein or to a specific region within the protein. For example, an “agent that specifically binds TGFβ, TNFα, IL-1β, IL-6, IL-1R, or IFNγ” includes antibodies and other agents that bind substantially to TGFβ, TNFα, IL-1β, IL-6, IL-1R, or IFNγ, respectively. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys).

Tumor necrosis factor (TNFα)-binding protein 1 (TBP1) refers to any form of TBP1. This is a soluble protein that is derived from a ubiquitous membrane receptor (e.g., tumor necrosis factor receptor 1) by proteolysis, and it binds TNFα. A tradename for TBP1 from receptor 1 is ONERCEPT®.

TNFα: Tumor necrosis factor (TNF, tumor necrosis factor α, TNFα, cachexin, or cachectin) is a cell signaling protein (cytokine) involved in systemic inflammation and is one of the cytokines that make up the acute phase reaction. It is produced chiefly by activated macrophages, although it can be produced by many other cell types, such as CD4+ lymphocytes, NK cells, neutrophils, mast cells, eosinophils, and neurons. Exemplary protein and nucleotide sequences for TNFα are available at GENBANK® (e.g., Accession Nos. P01375.1 and NM_001199054.1, respectively, incorporated by reference herein as available on Sep. 14, 2017).

Transforming growth factor beta-receptor (TGFβr1): Also known as TGFBR1 and activing receptor-like kinase 5 (ALK5; e.g., OMIM 190181), TGFβr1 is a serine/threonine kinase receptor for transforming growth factor-beta (TGFβ). Inhibitors of TGFβr1 have been developed, including the peptides P17 and P144 (see Patent Pub. No. US20120315256, incorporated herein by reference).

Transforming growth factor β (TGFβ): Also known as TGFB1 (e.g., OMIM 190180), TGFβ is a cytokine in the TGF superfamily Activated TGFβ activates different downstream substrates and regulatory proteins, inducing transcription of different target genes for cell differentiation, chemotaxis, proliferation, and activation of many immune cells. As TGFβ exhibits immunosuppressive functions, an increase in TGFβ expression often correlates with cancer malignancy, and disregulation of its immunosuppressive functions is implicated autoimmune diseases. Exemplary protein and nucleotide sequences for IFNγ are available at GENBANK® (e.g., Accession Nos. NP_000651.3 and NM_000660.6, respectively, incorporated by reference herein as available on Sep. 14, 2017).

Tear film: The layer of fluid including an aqueous layer covered by a lipid layer that covers the exposed area of the globe of the eye.

Therapeutic agent: The term “therapeutic agent” or “therapeutic,” when used in a generic sense, includes treating agents, prophylactic agents, and replacement agents.

Therapeutic protein: A protein that achieves a desired effect when administered to a subject, such as in the eye of the subject. A therapeutic protein can have a direct effect, such as a cytokine, or an indirect effect, such as Protein A, Protein G, Protein L or similar proteins found on the surface of Gram-positive bacteria, which can bind a monoclonal or polyclonal antibody that has a desired therapeutic effect.

Therapeutically effective amount: The term “therapeutically effective amount” refers to that amount of an active ingredient (such as a fusion protein) that is sufficient to effect treatment when administered to a mammal in need of such treatment. The therapeutically effective amount will vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or improving vision. The treatment may be assessed by objective or subjective parameters; including the results of a physical examination, neurological examination, or psychiatric evaluations.

Ulceration: Corneal ulcer, or ulcerative keratitis, is an inflammatory or more seriously, infective or non-infective condition of the cornea involving disruption of its epithelial layer with involvement of the corneal stroma.

Uveitis: Inflammation of the uvea, which is the pigmented layer that lies between the inner retina, and the outer fibrous layer, which is composed of the sclera and cornea. The uvea includes the middle layer of pigmented vascular structures of the eye with the iris, ciliary body, and choroid. Uveitis is an ophthalmic emergency and requires a thorough examination by an optometrist or ophthalmologist and urgent treatment to control the inflammation.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. A vector can be a viral vector.

von Willebrand factor (vWF): Also known as factor VIII-von Willebrand factor (F8VWF; e.g., OMIM 613160), vWF is a blood glycoprotein involved in hemostasis that includes a collagen-binding domain. vWF's primary function is binding to other proteins, in particular factor VIII, and it is important in platelet adhesion to wound sites. vWF has been shown to interact with collagen. vWF sequences are publicly available. For example, GENBANK® Accession Nos. AAB59458.1, AAP41950.1, and Q62935.2, incorporated by reference herein as available on Sep. 14, 2017, disclose exemplary human, rat, and mouse vWF protein sequences, and GENBANK® Accession Nos. NM_000552.4, AJ224673.1, and NM_011708.4, incorporated by reference herein as available on Sep. 14, 2017, disclose exemplary human, rat, and mouse vWF nucleotide sequences, respectively.

Wheat germ agglutinin (WGA): A lectin (carbohydrate-binding protein is a lectin that protects wheat (Triticum vulgaris) from insects, yeast and bacteria. It binds to N-acetyl-D-glucosamine and Sialic acid. In mammals the N-acetyl-D-glucosamine that WGA binds to is found in cartilage and the cornea, among other places, and sialic acid is found in mucous membranes (e.g., the lining of the inner nose and digestive tract). Examples of sequences are available in GENBANK® (e.g., Accession Nos. AAA34256.1 and M25536.1, incorporated herein by reference as available on Sep. 14, 207, which provide exemplary protein and nucleotide sequences for WGA, respectively); other examples are available herein (e.g., SEQ ID NO: 13).

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and, Harlow and Lane, Using Antibodies: A Laboratory Manual, Cold Spring Harbor Laboratory Press, 1999. In addition, the materials, methods, and examples are illustrative only and not intended to be limiting.

### II. FUSION PROTEIN

Local application of therapeutic compounds to the eye of a human subject is especially challenging because the compounds are washed out too quickly to have significant effects. Attaching a domain that anchors therapeutic proteins to the cornea so they are retained on the ocular surface and, therefore, can act for prolonged periods of time presents a versatile solution that can be applied to treat many conditions of the eye. Methods and compositions are disclosed herein for a fusion protein that includes an anchor domain and a therapeutic protein. The methods and compositions described herein include administering the fusion protein in a pharmaceutical composition to the eye of a subject.

The presently disclosed methods utilize fusion polypeptides comprising an anchor domain and a therapeutic protein. In some embodiments, the anchor protein includes a collagen-binding polypeptide, a heparin-binding polypeptide, or a lectin. In other embodiments, the therapeutic protein comprises a transforming growth factor beta (TGFβ) antagonist, a tumor necrosis factor alpha (TNFα) antagonist, an interleukin (IL)-1β antagonist, an IL-6 antagonist, an immunoglobulin-binding (Ig) polypeptide, an interferon (IFN)γ antagonist, IL-1Ra, or IL-10.

A. Anchor Domains

The disclosed fusion proteins include an anchor domain that allows the fusion protein to adhere to an ocular surface through non-covalent bonds. These anchor domains can bind collagen, heparin, heparan sulfate, or a carbohydrate. Exemplary collagen-binding anchor domains from the von Willebrand factor (vWF) are found in GENBANK® Accession Nos. AAB59458.1, AAP41950.1, and Q62935.2, incorporated by reference herein as available on Sep. 14, 2017, and as follows:

(SEQ ID NO: 1)
WREPSFMALS
(SEQ ID NO: 2)
WREPSFCALS

An exemplary collagen-binding anchor domain from Clostridium collagenase (ColH) are found in GENBANK® Accession No. BAA06251.1, incorporated by reference herein as available on Sep. 14, 2017, and as follows:

(SEQ ID NO: 3)
EIKDLSENKLPVIYMHVPKSGALNQKVVFYGKGTYDPDGSIAGYQWD
FGDGSDFSSEQNPSHVYTKKGEYTVTLRVMDSSGQMSEKTMKIKITD
PVYPIGTEKEPNNSKETASGPIVPGIPVSGTIENTSDQDYFYFDVIT
PGEVKIDINKLGYGGATWVVYDENNNAVSYATDDGQNLSGKFKADKP
GRYYIHLYMFNGSYMPYRINIEGSVGR

Exemplary heparin-binding (HS) anchor domains are as follows:

(SEQ ID NO: 4)
KRKKKGKGLGKKRDPSLRKYK
(SEQ ID NO: 5)
KRKKKGKGLGKKRDPCLRKYK

Carbohydrate-binding anchor domains include lectins and fragments thereof. Exemplary lectins are wheat germ agglutinin (WGA), concanavalin A (conA), and jacalin (Jac). An exemplary carbohydrate-binding anchor domain from ConA is:

(SEQ ID NO: 6)
MAISKKSSLFLPIFTFITMFLMVVNKVSSSTHETNALHFMFNQFSKDQK
DLILQGDATTGTDGNLELTRVSSNGSPQGSSVGRALFYAPVHIWESSAV
VASFEATFTFLIKSPDSHPADGIAFFISNIDSSIPSGSTGRLLGLFPDA
NVIRNSTTIDFNAAYNADTIVAVELDTYPNTDIGDPSYPHIGIDIKSVR
SKKTAKWNMQNGKVGTAHIIYNSVDKRLSAVVSYPNADSATVSYDVDLD
NVLPEWVRVGLSASTGLYKETNTILSWSFTSKLKSNEIPDIATVV
(GENBANK ® Accession No: CAA25787.1, incorporated
by reference herein as available on Sep. 14, 2017)

An exemplary carbohydrate-binding anchor domain from WGA is:

as available on Sep. 14, 2017)
(SEQ ID NO: 7)
MKMMSTRALALGAAAVLAFAAATAQAQRCGEQGSNMECPNNLCCSQ
YGYCGMGGDYCGKGCQNGACWTSKRCGSQAGGATCTNNQCCSQYGY
CGFGAEYCGAGCQGGPCRADIKCGSQAGGKLCPNNLCCSQWGFCGL
GSEFCGGGCQSGACSTDKPCGKDAGGRVCTNNYCCSKWGSCGIGPG
YCGAGCQSGGCDGVFAEAITANSTLLQE
(GENBANK ® Accession Nos.: AAA34256.1, AAA34258.1,
and AAA34257.1, incorporated by reference herein

An exemplary carbohydrate-binding anchor domain from Jac is:

(SEQ ID NO: 8)
MAYSSLLSLSVLALLFSISSADTRKWFLANGINQNPIGIIEAAVGVSED
LLNLNGMEAKNDEQSGISQTVIVGPWGAKVSTSSNGKAFDDGAFTGIRE
INLSYNKETAIGDFQVVYDLNGSPYVGQNHKSFITGFTPVKISLDFPSE
YIMEVSGYTGNVSGYVVVRSLTFKTNKKTYGPYGVTSGTPFNLPIENGL
IVGFKGSIGYWLDYFSMYLSL
(GENBANK ®: AAA32680.1, incorporated by reference
herein as available on Sep. 14, 2017)

One of skill in the art can readily identify fragments and variants of these anchor domains, see below. The anchor domain can be directly bound to a therapeutic polypeptide, or a linker can be inserted between the anchor domain and the therapeutic polypeptide. Exemplary polypeptides that includes linkers are set forth below:

(SEQ ID NO: 9)
GGGGS
(SEQ ID NO: 19)
EAAAKEAAAK

B. Therapeutic Polypeptides

The disclosed fusion proteins include a therapeutic polypeptide. In some embodiments, the therapeutic polypeptide can be, for example, a transforming growth factor beta (TGFβ) antagonist, a tumor necrosis factor alpha (TNFα) antagonist, an interleukin (IL)-1β antagonist, an IL-6 antagonist, an immunoglobulin-binding (Ig) polypeptide, an interferon (IFN)γ antagonist, IL-1Ra, or IL-10. In some non-limiting examples, the therapeutic polypeptide can be a P17, P144, or an antibody that specifically binds TGFβ, an inhibitor of TNFα polypeptide from the TNF receptor 1, an antibody that specifically binds TNFα, an antibody that specifically binds IL-1β, an antibody that specifically binds IL-6, an antibody that specifically binds IFNγ, protein A, protein G, and/or protein L.

One of skill in the art can readily identify therapeutic polypeptides of use. In some embodiments, the therapeutic protein is IL-10. An amino acid sequence for human IL-10 and a cDNA encoding this amino acid sequence are provided in GENBANK® Accession Nos. NP_000563.1 and CR542028, respectively, each of which are incorporated by reference herein as available on Sep. 14, 2017. A murine IL-10 and a cDNA encoding this amino acid sequence are provided in GENBANK® Accession Nos. EDL39722.1 and MUSIL10Z, respectively, each of which are incorporated by reference herein as available on Sep. 14, 2017, see also Fujio, K. et al., Adv Immunol, 105:99-130, 2010, incorporated herein by reference.

In some embodiments, the therapeutic protein is protein A. An exemplary protein A immunoglobulin-binding polypeptide is:

(SEQ ID NO: 10)
ADNKFNKEQQNAFYEILHLPNLNEEQRNGFIQSLKDDPSQSANLL
AEAKKLNDAQAPKADNKFNKEQQNAFYEILHLPNLTEEQRNGFIQ
SLKDDPSVSKEILAEAKKLNDAQAPK

Further, an amino acid sequence for Staphylococcus aureus protein A and a cDNA encoding this amino acid sequence are provided in GENBANK® Accession No. X61307.1, incorporated by reference herein as available on Sep. 14, 2017.

In some embodiments, the therapeutic protein is protein G. An exemplary protein G immunoglobulin-binding polypeptide is:

(SEQ ID NO: 11)
KTFTVTEKPEVIDASELTPAVTTYKLVINGKTLKGETTT
KAVDAETAEKAFKQYANDNGVDGVWTYDDATKTFTVTE

Further, an amino acid sequence for Streptococcus sp. GX7805 protein G and a cDNA encoding this amino acid sequence are provided in GENBANK® Accession No. Y00428.1, incorporated by reference herein as available on Sep. 14, 2017.

In some embodiments, the therapeutic protein is protein L. An amino acid sequence for Finegoldia magna protein L and a cDNA encoding this amino acid sequence are provided in GENBANK® Accession No. M86697.1, incorporated by reference herein as available on Sep. 14, 2017.

In some embodiments, the therapeutic protein is an inhibitor of TNFα polypeptide from the TNF receptor 1. An exemplary polypeptide sequence of this inhibitor is:

(SEQ ID NO: 12)
DSVCPQGKYIHPQNNSICCTKCHKGTYLYNDCPGPGQDTDCRECE
SGSFTASENHLRHCLSCSKCRKEMGQVEISSCTVDRDTVCGCRKN
QYRHYWSENLFQCFNCSLCLNGTVHLSCQEKQNTVCTCHAGHLRE
NECVSCSNCKKSLECTKLCLPQIEN

Further, an amino acid sequence for a human inhibitor of TNFα polypeptide from the TNF receptor 1 and a cDNA encoding this amino acid sequence are provided in GENBANK® Accession Nos. NP_001056 and NG_007506, respectively, incorporated by reference herein as available on Sep. 14, 2017.

In some embodiments, the therapeutic protein is an inhibitor of TGFβ. For example, the inhibitor can include amino acids 730 to 743 of the type III receptor of human TGFβ1. Amino acid sequences for human and rat inhibitors of TGFβ1 (such as P144) are provided in U.S. Pat. No. 7,582,609, which is incorporated herein by reference. For example, the protein can include the sequence TSLDASIIWAMMQN (SEQ ID NO: 13) Amino acid sequences for inhibitors of TGFβ1 (such as P17) are provided in U.S. Pat. Pub. No. 2010/022280, which is incorporated herein by reference. For example, the protein can include the sequence KRIWFIPRSSWYERA (SEQ ID NO: 14).

In some embodiments, the therapeutic protein is IL-1Ra. Exemplary sequences for IL-1Ra are known in the art. For example, protein and nucleotide sequences for IL-1Ra are available at GENBANK® (e.g., Accession Nos. CAA36262.1 and AJ005835.1, respectively, incorporated by reference herein as available on Sep. 18, 2017).

In some embodiments, the therapeutic protein is antibody that specifically binds TGFβ. These antibodies are known in the art. Exemplary amino acid sequences for antibodies that specifically bind TGFβ1 are provided in U.S. Pat. No. 7,527,791, which is incorporated herein by reference. In some embodiments, the therapeutic protein is antibody that specifically binds TNFα. Exemplary amino acid sequences for antibodies that specifically bind TNFα are provided in U.S. Pat. No. 6,258,562, which is incorporated herein by reference.

In some embodiments, the therapeutic protein is antibody that specifically binds IL-1β. These antibodies are known in the art. Exemplary amino acid sequences for antibodies that specifically bind IL-1β are provided in U.S. Pat. No. 8,623,367, which is incorporated herein by reference.

In some embodiments, the therapeutic protein is antibody that specifically binds IL-6. These antibodies are known in the art. Exemplary amino acid sequences for antibodies that specifically bind IL-6 are provided in U.S. Pat. No. 7,291,721, which is incorporated herein by reference. In some embodiments, the therapeutic protein is antibody that specifically binds IFNγ. Exemplary amino acid sequences for antibodies that specifically bind IFNγ are provided in U.S. Pat. No. 7,084,257, which is incorporated herein by reference.

Peptides that are similar to the anchor domains, therapeutic polypeptides, and linkers disclosed above can be used as well as fragments thereof that retain the therapeutic activity. These anchor domains and therapeutic polypeptides may contain substitutions, deletions, or additions. The differences are preferably in regions that are not significantly conserved among different species. Such regions can be identified by aligning the amino acid sequences of related proteins from various animal species. Generally, the biological effects of the peptide are retained. For example, a polypeptide at least 75%, 80%, 85%, 90%, 95%, 96%, 97%, 98% or 99% identical to one of these polypeptides can be utilized. Polypeptides are of use that include at most 1, 2, 3, 4, 5, 6, 7, 8, 9, or 10 conservative amino acid substitutions. Generally, polypeptides are of use provided they retain at least 75%, 80%, 85%, 90%, 95%, 96%, 97%, 98% or 99% of the biological function of the native polypeptide, or have increased biological function as compared to the native polypeptide.

In some embodiments, a fragment of the therapeutic protein is utilized. Generally a fragment of use retains at least 75%, 80%, 85%, 90%, 95%, 96%, 97%, 98% or 99% of the biological function of the native polypeptide, or has increased biological function as compared to the native polypeptide.

C. Fusion Proteins

A fusion protein of use in the methods disclosed herein includes at least two components: an anchor domain and a therapeutic protein. In some embodiments, the anchor domain can be attached to the therapeutic protein by a linker polypeptide, such as a GGGGS linker (SEQ ID NO: 9). In one embodiment, the therapeutic protein includes, in N-terminal to C-terminal order, the anchor domain and the therapeutic polypeptide. In another embodiment, the therapeutic protein includes, in N terminal to C-terminal order, the therapeutic polypeptide and the anchor domain. In either of these embodiments, a linker can be included between the anchor domain and the therapeutic polypeptide. Optionally, other components can be included at the N or C terminus, such as labels or a domain used for solubility and/or purification. In other embodiments, the fusion protein can include one or more copies of an anchor domain, therapeutic polypeptide (e.g., one of more copies of P144 or P17, such as at least about 1, 2, 3, 4, or 5 copies of P144 or P17 or about 2 copies of P144 or P17), or linker (e.g., one of more copies of SEQ ID NO: 9 or SEQ ID NO: 19, such as at least about 1, 2, 3, 4, or 5 copies of SEQ ID NO: 9 or SEQ ID NO: 19 or about 2 copies of SEQ ID NO: 9 or SEQ ID NO: 19). Additional information on therapeutic polypeptides and fusion proteins is provided below.

Also encompassed herein are therapeutic polypeptides that are fused to a heterologous peptide, such as a peptide that can be used for detecting; purifying; stabilizing; or solubilizing the polypeptide. In particular non-limiting examples, a solubilization domain can be included, to enhance solubility. Suitable solubilization domains include a maltose binding protein and the small ubiquitin-like modifier (SUMO).

In some embodiments, the fusion protein includes a collagen-binding domain, such as a collagen-binding polypeptide from von Willebrand factor (vWF) or Clostridium collagenase (ColH) and a therapeutically effective, Ig-binding polypeptide from protein A, protein G and/or protein L. The Ig-binding polypeptide can include the entire protein A, protein G and/or protein L or an Ig-binding fragment thereof. Exemplary polypeptides that include a protein G polypeptide as the Ig-binding polypeptide and a vWF polypeptide as the anchor domain are as follows:

(SEQ ID NO: 15; linkers are underlined)
MWREPSFMALSASGGGGSGGGGSASMGTPAVTTYKLVINGKT
LKGETTTKAVDAETAEKAFKQYANDNGVDGVWTYDDATKTFT
VTEVNTPAVTTYKLVINGKTLKGETTTKAVDAETAEKAFKQY
ANDNGVDGVWTYDDATKTFTVTEIGENLYFQGIDENLYFQGG
SHHHHHHH
(SEQ ID NO: 16, linkers are underlined)
MSKKHHHHHHHHGGGGSASMGTPAVTTYKLVINGKTLKGETT
TKAVDAETAEKAFKQYANDNGVDGVWTYDDATKTFTVTEVNT
PAVTTYKLVINGKTLKGETTTKAVDAETAEKAFKQYANDNGV
DGVWTYDDATKTFTVTEIGENLYFQGIDENLYFQGGSAGGGG
SGGGGSASWREPSFMALS

In other embodiments, the therapeutically effective polypeptide can be a polypeptide from an inhibitor of TNFα polypeptide from the TNF receptor 1 or an inhibitor of TGFβ. The polypeptide from an inhibitor of TNFα polypeptide from the TNF receptor 1 or an inhibitor of TGFβ can include the entire inhibitor protein or a therapeutically effective fragment thereof. In some examples, the anchor domain can be a vWF polypeptide, and the therapeutic polypeptide can be an inhibitor of TGFβ. Exemplary polypeptides that include tandem repeats of P144 and P17, respectively, as the inhibitor and a vWF polypeptide as the anchor domain are as follows:

(SEQ ID NO: 17; linkers are underlined)
MSKKWREPSFMALSASGGGGSGGGGSEAAAKTSLDASIIWA
MMQNEAAAKEAAAKTSLDASIIWAMMQNGGGGSHHHHHHHH
(SEQ ID NO: 18; linkers are underlined)
MSKKWREPSFMALSASGGGGSGGGGSGGGGSKRIWFIPRSSW
YERAGGGGSGGGGSKRIWFIPRSSWYERAGGGGSHHHHHHHH

In additional embodiments, the fusion protein includes a collagen-binding domain, such as a collagen-binding polypeptide from von Willebrand factor (vWF) or Clostridium collagenase (ColH) or a heparin sulfate-binding domain and a therapeutically effective polypeptide from IL-10. The polypeptide from IL-10 can include the entire IL-10 protein or a therapeutically effective fragment thereof.

Also included are peptide derivatives of the anchor domains, therapeutically effective polypeptide, and/or fusion protein, which are differentially modified during or after synthesis, such as by benzylation, glycosylation, acetylation, phosphorylation, amidation, pegylation, derivatization by known protecting/blocking groups. In some embodiments, peptides can include at least one amino acid or every amino acid that is a D stereoisomer. Other peptides can include at least one amino acid that is reversed. The amino acid that is reversed may be a D stereoisomer. Every amino acid of a peptide may be reversed and/or every amino acid can be a D stereoisomer.

Any of the disclosed anchor domains, therapeutically effective polypeptides, or fusion proteins can be readily synthesized by automated solid phase procedures well known in the art. Techniques and procedures for solid phase synthesis are described in Solid Phase Peptide Synthesis: A Practical Approach, by E. Atherton and R. C. Sheppard, published by IRL, Oxford University Press, 1989. Alternatively, these peptides may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). Other methods useful for synthesizing peptides of the present disclosure are described in Nakagawa et al., J. Am. Chem. Soc. 107:7087-7092, 1985.

D. Polynucleotides and Host Cells

Polynucleotides encoding the fusion proteins disclosed herein are also provided. These polynucleotides include DNA, cDNA and RNA sequences which encode the fusion protein. The coding sequence includes variants that result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W. H. 5 Freeman and Co., NY).

A nucleic acid encoding the fusion protein can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well-known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

A polynucleotide sequence encoding the fusion protein can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, a ribosome binding site, transcription terminators, transcriptional regulators (e.g., AraC and LacI) a start codon (e.g., ATG) in front of a protein-encoding gene, splicing signal for introns, maintenance of the correct reading frame of that gene to permit proper translation of mRNA, and stop codons.

The polynucleotides encoding the fusion protein include a recombinant DNA, which is incorporated into a vector in an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

In one embodiment, vectors are used for expression in yeast such as Saccharomyces cerevisiae or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2 μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K lactis are known, such as pKLAC1. Thus, in one example, after amplification in bacteria, plasmids can be introduced into the corresponding yeast auxotrophs by methods similar to bacterial transformation. The polynucleotides can also be designed to express in insect cells.

The fusion protein can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared encoding the fusion protein disclosed herein. A number of viral vectors have been constructed, including polyoma, SV40 (Madzak et al., J. Gen. Virol., 73:15331536, 1992), adenovirus (Berkner, Cur. Top. Microbiol. Immunol., 158:39-6, 1992; Berliner et al., Bio Techniques, 6:616-629, 1998; Gorziglia et al., J. Virol., 66:4407-4412, 1992; Quantin et al., Proc. Nad. Acad. Sci. USA, 89:2581-2584, 1992; Rosenfeld et al., Cell, 68:143-155, 1992; Wilkinson et al., Nucl. Acids Res., 20:2233-2239, 1992; Stratford-Perricaudet et al., Hum. Gene Ther., 1:241-256, 1990), vaccinia virus (Mackett et al., Biotechnology, 24:495-499, 1992), adeno-associated virus (Muzyczka, Curr. Top. Microbiol. Immunol., 158:91-123, 1992; On et al., Gene, 89:279-282, 1990), herpes viruses including HSV and EBV (Margolskee, Curr. Top. Microbiol. Immunol., 158:67-90, 1992; Johnson et al., J. Virol., 66:29522965, 1992; Fink et al., Hum. Gene Ther. 3:11-19, 1992; Breakfield et al., Mol. Neurobiol., 1:337-371, 1978; Fresse et al., Biochem. Pharmacol., 40:2189-2199, 1990), Sindbis viruses (H. Herweijer et al., Human Gene Therapy, 6:1161-1167, 1995; U.S. Pat. Nos. 5,091,309 and 5,2217,879), alphaviruses (S. Schlesinger, Trends Biotechnol. 11:18-22, 1993; I. Frolov et al., Proc. Natl. Acad. Sci. USA, 93:11371-11377, 1996) and retroviruses of avian (Brandyopadhyay et al., Mol. Cell Biol., 4:749-754, 1984; Petropouplos et al., J. Virol., 66:3391-3397, 1992), murine (Miller, Curr. Top. Microbiol. Immunol., 158:1-24, 1992; Miller et al., Mol. Cell Biol., 5:431-437, 1985; Sorge et al., Mol. Cell Biol., 4:1730-1737, 1984; Mann et al., J. Virol., 54:401-407, 1985), and human origin (Page et al., J. Virol., 64:5370-5276, 1990; Buchschalcher et al., J. Virol., 66:2731-2739, 1992). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding a fusion protein is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, and poliovirus vectors.

DNA sequences encoding the fusion protein can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts cells also can include microbial, insect, and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), Methods in Enzymology: Cell Culture, volume 58, Academic Press, Inc., Harcourt Brace Jovanovich, N.Y., 1979). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods, Methods in Enzymology, 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a C-terminal endostatin polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see, for example, Eukaryotic Viral Vectors, Cold Spring Harbor Laboratory, Gluzman ed., 1982).

E. Chemical Conjugation

While molecular methods can be used to synthesize fusion proteins, chemical methods can alternatively be used to synthesize fusion protein by linking an anchor domain to a therapeutic polypeptide. In some examples, an anchor domains (e.g., a lectin carbohydrate-binding domain, such as WGA, conA, and Jac, or a collagen- or heparin-binding domain) can be covalently bound to a therapeutic polypeptide (e.g., an Ig-binding polypeptide or a TGFβ antagonist, a TNFα antagonist, an IL-1β antagonist, an IL-6 antagonist, or an IFNγ antagonist) through chemical conjugation. Various types of chemical reagents can be used (see, e.g., Ido et al., JBC, 287(31): 26377-26387, 2012, and U.S. Pat. Pub. No. 2003/0040496, both incorporated herein by reference). In some examples, 1-ethyl-3-(3-dimethylaminopropyl)carbodiimide hydrochloride (EDC) is used for chemical conjugation (see, e.g., Ido et al., JBC, 287(31): 26377-26387, 2012, incorporated herein by reference). In some non-limiting examples, at least about 1-2, 2-5, 5-10, 10-15, 15-20, 20-25, or 25-30 mM or at least about 2, 5, 6, 10, 15, 20, or 25 mM EDC can be used. In further non-limiting embodiments, N-hydroxysulfosuccinimide (Sulfo-NHS) can be used in addition to the ECD (see, e.g., Ido et al., JBC, 287(31): 26377-26387, 2012, incorporated herein by reference). In some examples, at least about 5-10, 10-20, 20-30, 30-40-, 40-50, 50-75, 75-100, or 100-200 mM or at least about 5, 10, 25, 50, or 100 mM Sulfo-NHS can be used in addition to the ECD.

In other embodiments, alternative chemical conjugation (i.e., cross-linking) reagents may be used to form covalent bonds between amino groups and thiol groups and to introduce thiol groups into proteins (see, e.g., U.S. Pat. Pub. No. 2003/0040496, incorporated herein by reference). Additional alternative chemical conjugation (i.e., cross-linking) reagents can be found in the PIERCE CATALOG, ImmunoTechnology Catalog & Handbook, 1992-1993, which describes the preparation of and use of such reagents and provides a commercial source for such reagents (incorporated herein by reference; see also, e.g., Cumber et al., Bioconjugate Chem. 3:397-401, 1992; Thorpe et al., Cancer Res. 47:5924-5931, 1987; Gordon et al., Proc. Natl. Acad Sci. 84:308-312, 1987; Walden et al., J. Mol. Cell Immunol. 2:191-197, 1986; Carlsson et al., Biochem. J. 173:723-737, 1978; Mahan et al., Anal. Biochem., 162:163-170, 1987; Wawryznaczak et al., Br. J. Cancer 66:361-366, 1992; Fattom et al., Infection & Immun 60:584-589, 1992, all of which are incorporated herein by reference). These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-α-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfosuccinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl- -methyl- -(2-pyridylthio)toluene (SMPT, hindered disulfate linker); sulfosuccinimidyl 6[-methyl- -2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinmidyl(4-iodoacetyl)aminobenzoate (SIAB; thioether linker); sulfosuccinimidyl(4-iodoacetyl)amino benzoate (sulfo-SIAB); succinimidyl4(p-maleimidophenyl)butyrate (SMPB); sulfosuccinimidyl4-(p-maleimidophenyl)butyate (sulfo-SMPB); and azidobenzoyl hydrazide (ABH).

### III. PHARMACEUTICAL COMPOSITIONS AND METHODS OF TREATMENT

Compositions and methods are disclosed herein for treating a subject, such as a mammalian subject (e.g., a human or veterinarian subject), with a disorder that affects the eye. In some examples, the methods can ameliorate a sign or symptom of a disorder that affects the eye (e.g., corneal haze or scarring, dry eye, and/or inflammation at an ocular surface of the eye) in a subject. Administering the fusion protein is sufficient to treat, inhibit, and/or prevent the disorder that affects the eye.

The methods include administering a therapeutically effective amount of a fusion protein that includes an anchor domain and a therapeutic polypeptide. In some examples, the anchor domain can be a carbohydrate-binding anchor domain from a lectin. A carbohydrate-binding anchor domain from any lectin that specifically binds an ocular surface of the eye (e.g., the cornea) can be used (see, e.g., Uusitalo et al., Histochemical Journal, 26: 787-798, 1994, incorporated herein by reference). Non-limiting examples of lectins can include wheat germ agglutinin (WGA), jacalin (jac), and concanavalin A (conA). In other examples, the anchor domain is a collagen-binding anchor domain from von Willebrand factor (vWF) or Clostridium collagenase (ColH). In other examples, the anchor domain can be a heparin-binding anchor domain.

In some examples, the therapeutic polypeptide can be a transforming growth factor beta (TGFβ) antagonist, a tumor necrosis factor alpha (TNFα) antagonist, an interleukin (IL)-1β antagonist, an IL-6 antagonist, an immunoglobulin-binding (Ig) polypeptide, an interferon (IFN)γ antagonist, or IL-10, interleukin-1 receptor antagonist (IL-1Ra) or an effective fragment or variant thereof. In some non-limiting examples, the TGFβ antagonist can be P17, P144, or an antibody that specifically binds TGFβ. In other examples, the TNFα antagonist can be an inhibitor of TNFα polypeptide from the TNF receptor 1 or an antibody that specifically binds TNFα. In further examples, the IL-1β antagonist can be an antibody that specifically binds IL-1β. In additional examples, the IL-6 antagonist can be an antibody that specifically binds IL-6. In some other examples, the IFNγ antagonist can be an antibody that specifically binds IFNγ. In still further examples, the Ig-binding polypeptide can be protein A, protein G, or protein L or an effective fragment or variant thereof.

In further embodiments, where an Ig-binding polypeptide is administered as a therapeutic polypeptide, an additional therapeutically effective amount of an antibody is administered. In some examples, the antibody can be an antibody that specifically binds TGFβ, TNFα, IL-1β, IL-6, or IFNγ.

In some embodiments, the methods include administering a fusion protein that includes a collagen-binding anchor domain from von Willebrand factor (vWF) or the ColH collagen-binding anchor domain and a TGFβ antagonist, an Ig-binding polypeptide, the inhibitor of TNFα polypeptide from the TNF receptor 1, IL-1Ra, or IL-10. In certain examples, the TGFβ antagonist can be P17 or p177. In other examples, the Ig-binding polypeptide can be protein A, protein G, or protein L or an effective fragment thereof.

In other embodiments, the methods include administering a fusion protein that includes a carbohydrate-binding anchor domain from WGA as an anchor domain and a TGFβ antagonist, a TNFα antagonist, an IL-1β antagonist, an IL-6 antagonist, an interferon (IFN)γ antagonist, the inhibitor of TNFα polypeptide from the TNF receptor 1, IL-10, or IL-1Ra as a therapeutic polypeptide. In some non-limiting examples, the TGFβ antagonist can be an antibody that specifically binds TGFβ. In other examples, the TNFα antagonist can be an antibody that specifically binds TNFα. In further examples, the IL-1β antagonist can be an antibody that specifically binds IL-1β. In additional examples, the IL-6 antagonist can be an antibody that specifically binds IL-6. In some other examples, the IFNγ antagonist can be an antibody that specifically binds IFNγ.

In further embodiments, the methods include administering a fusion protein that includes a heparin-binding anchor domain as an anchor domain and a TNFα antagonist. In some non-limiting examples, the TNFα antagonist can be an inhibitor of TNFα polypeptide from the TNF receptor 1.

In additional embodiments, a subject is selected that has or is at risk for at least one disorder that affects the eye. The subject can have a disorder in one or both eyes, and the disorder can be a chronic or an acute disorder. Methods for selecting subjects with a disorder of the eye are known in the medical arts and can be used to select the subjects described herein. In some non-limiting examples, the disorder can be corneal haze or scarring, dry eye, and/or inflammation. The methods include administering the fusion protein to the subject with the disorder that affects the eye. In some embodiments, the methods include selecting a subject with corneal haze or scarring (e.g., U.S. Pat. No. 6,143,315 and U.S. Patent Pub No. 2003/0153524, incorporated herein by reference). Any part of the cornea tissue affected by corneal haze or scarring can be treated. In some non-limiting examples, the middle layer of the cornea (i.e., the stroma) is affected. Further, the corneal haze or scarring can be due to any condition. In non-liming examples, the corneal haze can be due to trauma, infection, or surgery. In other non-limiting examples, the corneal scarring is due to injury. In certain embodiments, the injury that causes the corneal scarring is abrasion, laceration, a burn, or a disease.

In some non-limiting embodiments where the subject has corneal haze or scarring, the methods include administering a fusion protein that includes a TGFβ antagonist or administering a fusion protein that includes an immunoglobulin-binding (Ig) polypeptide and an antibody that specifically binds TGFβ. In some non-limiting examples, the TGFβ1 antagonist can be P17, P144, or an antibody that specifically binds TGFβ. In some other examples, the Ig-binding polypeptide can be an antibody-binding polypeptide of protein A, protein G, or protein L. In some non-limiting embodiments, the methods include administering a fusion protein that includes a collagen-binding anchor domain from von Willebrand factor (vWF) and a TGFβ antagonist (e.g., P17 and P144) or an Ig-binding polypeptide and an antibody that specifically binds TGFβ. In other non-limiting embodiments, the methods include administering a fusion protein that includes a carbohydrate-binding anchor domain from WGA as an anchor domain and a TGFβ antagonist (e.g., an antibody that specifically binds TGFβ). In some examples, the method can be used to reduce or ameliorate signs and symptoms of corneal haze and/or scarring. In some non-limiting examples where a subject has corneal haze, the method can be used to reduce or ameliorate blurry, unfocused, or obscured vision and/or visible halos around light. In other non-limiting examples, where the subject has corneal scarring, the methods can be used to reduce or ameliorate blurred vision or total blindness.

In some embodiments, the methods include selecting a subject with dry eye disease (DED; see, e.g., U.S. Patent Pub. No. 2006/0281739, incorporated herein by reference). The DED can be caused by any condition. In some non-limiting examples, the DED can be caused by keratoconjunctivitis sicca, Sjörgen's syndrome, corneal injury, age-related dry eye, Stevens-Johnson syndrome, congenital alachrima, pharmacological side effects, infection, Riley-Day syndrome, conjunctival fibrosis, eye stress, glandular and tissue destruction, ocular cicatrical pemphogoid, blepharitis, autoimmune and other immunodeficient disorders, allergies, diabetes, lacrimal gland deficiency, lupus, Parkinson's disease, Sjögren's syndrome, rheumatoid arthritis, rosacea, environmental exposure to excessively dry air, airborne particulates, smoke, smog, and/or an inability to blink.

In some embodiments, the subject can have Sjögren's syndrome. Dry eye is a hallmark of Sjögren's syndrome; the disease is highly recalcitrant to current therapies and is commonly associated with a significant decline in quality of life (Le, Q et al., BMC Opthalmol, 12:22, 2012; Li, M et al., Invest Opthalmol Vis Sci, 53:5722-5727, 2012). In severe disease, loss of ocular surface integrity provoked by chronic inflammation can be further complicated by sight-threatening consequences, such as keratinization, scarring, ulceration, and infection (Foulks, G. N. et al., Ocul Surf, 13:118-132, 2015; McNamara, N. A., Optom Vis Sci, 87:233-238, 2010; Stern, M. E. et al., Int Rev Immunol, 32:19-41, 2013). Present treatment options, such as the widely-used artificial tear supplements can provide modest and temporary relief of symptoms but do not target disease mechanisms; for example, cyclosporine A, an FDA-approved ophthalmic formulation for DED (dry eye disease), benefits just 15% of patients (versus 5% with placebo) (Sall, K. et al., Ophthalmology, 107:631-639, 2000; Ames, P. et al., Clin Investig (Lond), 5:267-285, 2015). The presently disclosed methods can be used in combination with other treatments for Sjögren's syndrome, such as artificial tears or steroids.

In further embodiments, a subject with inflammation of the eye (e.g., chronic inflammation of the eye) is selected for treatment. In some examples, the inflammation can be caused by glaucoma, uveitis, macular degeneration, diabetic retinopathy, other retinal problems, infection (e.g., bacterial, fungal, viral, or parasitic infection, such as blepharitis, chalazion, conjunctivitis, dacryocystitis, iritis, keratitis, periorbital cellulitis, scleritis, sinusitis, and stye or bordeolum), allergy (e.g., drug allergies, food allergies, hay fever or allergic reaction to an allergen, and insect bite allergy, including chronic or acute allergies), autoimmune disorders (e.g., ankylosing spondylitis, Behcet's syndrome, dermatomyositis, Graves' disease, juvenile rheumatoid arthritis, multiple sclerosis, psoriatic arthritis, Reiter's syndrome, rheumatoid arthritis, Sjögren's syndrome, systemic lupus erythematosus, and Wegener's granulatomatosis), graft versus host disease, dry eye syndrome, limbal stem cell insufficiency, irritation, and injury or trauma to the eye or eyelid, such as due to blunt trauma, corneal abrasion or ulcer, foreign objects or materials, hematoma, insect bite or sting, irritants, and orbital bone fracture (see U.S. Pat. Pub. No. 2013/0336557, incorporated herein by reference). In other embodiments, the subject can have or be at risk for keratinization, scarring, ulceration, and infection due to inflammation (e.g., chronic inflammation).

Where a subject has DED and/or inflammation (e.g., chronic inflammation) of the eye, the methods include administering a fusion protein that includes a tumor necrosis factor alpha (TNFα) antagonist, an interleukin (IL)-1β antagonist, an IL-6 antagonist, an interferon (IFN)γ antagonist, IL-10, or IL-1Ra or administering a fusion protein that includes an immunoglobulin-binding (Ig) polypeptide and an antibody that specifically binds TNFα, IL-1β, IL-6, or IFNγ. In some examples, the Ig-binding polypeptide can be an antibody-binding polypeptide from protein A, protein G, or protein L or an Ig-binding fragment thereof. In certain non-limiting examples, the TNFα antagonist comprises an inhibitor of TNFα polypeptide from the TNF receptor 1 or an antibody that specifically binds TNFα. In other examples, the IL-1β antagonist comprises an antibody that specifically binds IL-1β. In additional examples, the IL-6 antagonist comprises an antibody that specifically binds IL-6. In still further examples, the IFNγ antagonist comprises an antibody that specifically binds IFNγ. In certain non-limiting embodiments, the methods include administering a fusion protein that includes a collagen-binding anchor domain from von Willebrand factor (vWF) and an Ig-binding polypeptide as well as an additional therapeutically effective amount of an antibody (e.g., an antibody that specifically binds TNFα, IL-1β, IL-6, or IFNγ). In additional non-limiting embodiments, the methods include administering a fusion protein that includes a collagen-binding anchor domain from von Willebrand factor (vWF) and the inhibitor of TNFα polypeptide from the TNF receptor 1, IL-10, or IL-1Ra. In other non-limiting embodiments, the methods include administering a fusion protein that includes a carbohydrate-binding anchor domain from WGA as an anchor domain and a TNFα antagonist (e.g., an antibody that specifically binds TNFα or the inhibitor of TNFα polypeptide from the TNF receptor 1), an IL-1β antagonist (e.g., an antibody that specifically binds IL-1β), an IL-6 antagonist (e.g., an antibody that specifically binds IL-6), an interferon IFNγ antagonist (e.g., an antibody that specifically binds IFNγ), IL-10, or IL-1Ra as a therapeutic polypeptide. In some examples, the disclosed methods can be used to reduce or ameliorate signs or symptoms of DED and/or inflammation of the eye. In some examples where the subject has DED, the methods can be used to reduce or ameliorate a burning sensation, itchy eyes, aching sensations, heavy eyes, fatigued eyes, sore eyes, a dryness sensation, red eyes, photophobia (light sensitivity), blurred vision, foreign body sensation (i.e., a feeling that grit or some other object or material is in the eye), and watery eyes. In some examples where the subject has inflammation (e.g., chronic inflammation), the methods can be used to reduce or ameliorate itching, excessive tear production, eye discharge, pain, redness, swelling, tearing, and/or unusual warmth or heat (see U.S. Pat. Pub. No. 2013/0336557, incorporated herein by reference).

In some embodiments, the fusion protein is administered locally. Local modes of administration include topical application, such as eye drops, or any other pharmaceutical dosage form formulated for administration to the eye. Administration can include an extended release formulation, such as by dissociation of the fusion protein or, if collagen is bound, though digestion of collagen by collagenases. In an embodiment, significantly smaller amounts of the therapeutic polypeptide (compared with systemic approaches) may exert an effect when administered locally (for example, topically to the cornea) compared to when the therapeutic polypeptide is administered systemically (for example, intravenously). In some embodiments, the disclosed compositions are administered topically to the conjunctiva, cornea, and/or anterior chamber.

In some embodiments, administration is from an ocular delivery device that is specifically adapted for delivery to the eye or a subcompartment thereof. For example, the device can be made of a sterile and/or biologically inert material configured for implantation in the eye. The device may use an internal reservoir (for example, from an implant disposed at an intra- or extra-ocular location (see, U.S. Pat. Nos. 5,443,505 and 5,766,242)) or from an external reservoir (for example, from an intravenous bag). A variety of devices suitable for administering components locally to the inside of the eye are known in the art. See, for example, U. S. Pat. Nos. 6,251,090, 6,299,895, 6,416,777, 6,413,540, and PCT Publication No. PCT/US00/28187. Methods for administration of agents to the eye are known in the medical arts and can be used to administer components described herein.

A suitable topical formulation of a fusion protein containing a carbohydrate-binding anchor domain from a lectin, collagen-binding anchor domain from von Willebrand factor (vWF) or Clostridium collagenase (ColH), or a heparin-binding anchor domain and a therapeutic polypeptide that includes a transforming growth factor beta (TGFβ) antagonist, a tumor necrosis factor alpha (TNFα) antagonist, an interleukin (IL)-1β antagonist, an IL-6 antagonist, an immunoglobulin-binding (Ig) polypeptide, an interferon (IFN)γ antagonist, IL-1Ra, or IL-10 or an effective fragment or variant thereof and is, for example, eye drops containing about, for example, about 0.1, 1, 10, 100, 1,000, or 10,000 μM of the fusion protein. In some embodiments, the fusion protein can be administered at a dose in the range of about 0.1, 1, 10, 100, 1,000, or 10,000 μM per day, either as a single dose or as divided doses. In a specific non-limiting example, the dose is administered about 1, 2, or 3 times daily. In some embodiments, the fusion protein is administered for at least 1, 2, 3, 4, 5, 6, 7, 8, 9, 10, 11, 12, 13, or 14 days or 3 or 4 weeks.

A pharmaceutical composition can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the principal active ingredients, the vehicles and compositions can include various formulatory ingredients, such as anti-microbial preservatives and tonicity agents. For example, antimicrobial preservatives include: benzalkonium chloride, thimerosal, chlorobutanol, methylparaben, propylparaben, phenylethyl alcohol, EDTA, sorbic acid, POLYQUAD® and other agents equally well known to those skilled in the art. Such preservatives, if employed, will typically be used in an amount from about 0.0001 wt. % to 1.0 wt. %. Suitable agents which may be used to adjust tonicity or osmolality of the compositions include mannitol, dextrose, glycerine and propylene glycol. If used, such agents will be employed in an amount of about 0.1 wt. % to 10.0 wt. %. However, the composition does not include preservatives or tonicity agents which are known to adversely affect or irritate the eye, such as the cornea.

In addition to the active ingredient, a pharmaceutical composition can further comprise one or more additional pharmaceutically active agents. Controlled- or sustained-release formulations of a pharmaceutical composition of the invention may be made using conventional technology.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.01% to about 10% (w/w) active ingredient, although the concentration of the active ingredient may be as high as the solubility limit of the active ingredient in the solvent. Formulations for topical administration may further comprise one or more of the additional ingredients described herein.

The pharmaceutical compositions may be prepared, packaged, or sold in the form of a sterile aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parenterally administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for ophthalmic administration. Such formulations may, for example, be in the form of eye drops including, for example, a 0.01% to 10% (w/w) solution or suspension of the active ingredient in an aqueous or oily liquid carrier. Such drops may further comprise buffering agents, salts, or one or more other of the additional ingredients described herein. Other administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., Remington's Pharmaceutical Sciences, Mack Publishing Co., Easton, Pa., 1985, and U.S. Pat. No. 6,534,059, which is incorporated herein by reference. In some embodiments, additional pharmaceutically active drugs can be included in the vehicles of to make ophthalmic compositions. Drugs which can be delivered in vehicles include, but are not limited to, steroids, growth factors, antioxidants, aldose reductase inhibitors, non-steroidal anti-inflammatories, immunomodulators, anti-allergics, antimicrobials, and beta-blockers.

Dosage treatment may be a single dose schedule or a multiple dose schedule to ultimately deliver the amount specified above. The doses can be intermittent. Moreover, the subject may be administered as many doses as appropriate. In some embodiments, the subject is administered a fusion protein prior to the onset of a condition. Administration may be provided as a single administration or a periodic bolus. Thus, the disclosed compositions can be administered hourly, 1, 2, 3, or 4 times daily, daily, every other day, or weekly. The disclosed compositions can be administered when the subject experiences a “flare-up” of the disease condition.

Individual doses are typically not less than an amount required to produce a measurable effect on the subject, and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount, which can be adjusted for local applications. Effective amounts of dose and/or dose regimen can readily be determined empirically from preclinical assays, from safety and escalation and dose range trials, individual clinician-patient relationships, as well as in vitro and in vivo assays. Generally, these assays will evaluate inflammation, the rate of tear formation, the integrity of the corneal epithelium by staining with dyes, or expression of a biological component (cytokine, specific inflammatory cell, microglia, etc.) that affects inflammation or the rate of tear formation.

In some embodiments, the method results in a therapeutic benefit, such as preventing the development of, halting the progression of, and/or reversing the progression of a disorder of the eye and/or inflammation. The subject can have any form of a disorder of the eye, such as, but not limited to, dry eye, Sjögren's syndrome, corneal haze, or corneal scarring. The subject can have any form of inflammation of the eye, such as inflammation caused by glaucoma, uveitis, macular degeneration, diabetic retinopathy, other retinal problem, or a bacterial or viral infection, such as a bacterial keratitis infection. The therapeutic benefit can reduce the risk of keratinization, scarring, ulceration, and infection due to chronic inflammation.

In some embodiments, the method includes the step of detecting that a therapeutic benefit has been achieved. Measures of therapeutic efficacy will be applicable to the particular disease being modified, and will recognize the appropriate detection methods to use to measure therapeutic efficacy. Suitable tests are disclosed below, and include tests for the presence of dry eye, such as Schirmer's test or by measuring lactoferrin or lysozyme content or the rate at which eye drops containing fluorescein or other type of dye are flushed out of the eye. In other embodiments, measures of therapeutic efficacy include measuring cornea epithelial damage using fluorescein, LISSAMINE™ green or other dyes.

The disclosure is illustrated by the following non-limiting Examples.

### VI. EXAMPLES

The following examples are provided to illustrate particular features of certain embodiments, but the scope of the claims should not be limited to those features exemplified.

### Example 1

This example discloses using a TNFα trap with an anchor domain to inhibit TNFα in vitro. Large amounts of a binding protein are immobilized to provide a sink for an extracellular signaling molecule. Tumor necrosis factora (TNFα) is a potent mediator of inflammation that is strongly upregulated by LPS in the cornea (Sekine-Okano, M. et al., Invest Ophthalmol Vis Sci, 37:1302-1310, 1996; Gupta, D. et al., Invest Ophthalmol Vis Sci, 53:6589-6599, 2012; Vij, N. et al., Invest Ophthalmol Vis Sci, 46:88-95, 2005; Kalliolias, G. D. et al., Nat Rev Rheumatol, 12:49-62, 2016; Waters, J. P. et al., J Pathol, 230:132-147, 2013). A sink is created for TNFα using a fusion of the TNFα binding protein TBP1 (Ythier, A. et al., Cytokine, 5:459-462, 1993; Lou, J. et al., J Neuroimmunol, 77:107-115, 1997; Sakimoto, T. et al., Invest Ophthalmol Vis Sci, 55:2419-2423, 2014) with an anchor domain that binds to collagen (abbreviated TBP1_{COL})

An amount of TNFα that is sufficient to kill 70-80% of L929 fibroblasts is pre-incubated with various amounts of TBP1_{COL}, and then TNFα activity towards the fibroblasts is assayed as described (Shiau, M. Y. et al., Mediators Inflamm, 10:199-208, 2001). For comparison, commercial untagged TBP1 (R&D Systems) is titrated in parallel. Some of the TNFα protein is labeled with ALEXA FLUOR® 488 amino reactive dye (ThermoFischer Scientific) (Huang, S. et al., Electrophoresis, 25:779-784, 2004) while still attached to the resin (see Example 9). The activity of the labeled TBP1_{COL} to neutralize TNFα activity is tested as above.

Results: TBP1_{COL} has two functionalities. Function of the effector domain is assayed based on its ability to neutralize TNFα at a concentration similar to commercial TBP1 and consistent with a high binding affinity. The function of the anchor domain is assayed based on binding to immobilized collagen III (McKenna, S. D. et al., J Pharmacol Exp Ther, 322:822-828, 2007).

### Example 2

This example discloses using a TNFα trap with an anchor domain to inhibit LPS-induced inflammation in vivo.

In vivo model: Mice are anesthetized by intraperitoneal injection of ketamine (50 mg/kg) and xylazine (5 mg/kg), and wounds are made with an AlgerBrush II as previously described (Basu, S. et al., Sci Transl Med, 6:266ra172, 2014). One drop of proparacaine hydrochloride (0.5%) is added to each eye just before debridement, and 20 μg LPS from P. aeruginosa (Sigma-Aldrich) in 2 μl phosphate-buffered saline (PBS) is applied to the wounded corneas. Commercial LPS is contaminated; therefore, LPS is purified as previously described (Brothers, K. M. et al., Sci Rep, 5:14003, 2015).

The TBP1_{COL} dose: The dose required for maximal binding is assayed by adding increasing amounts (0.1-10⁴ μg/ml) of fluorescently labeled TBP1_{COL} to the corneas of two mice, and the relative amounts of bound protein are analyzed by photographing with dissecting microscope equipped with a blue light source and a digital camera at a fixed distance. The relative amounts of label in the eyes are determined with the ImageJ program.

Time-course of the effects of a single dose of TBP1_{COL}: Four mice are treated with vehicle, and 4 mice are treated with fluorescently labeled TBP1_{COL} 3-4 hours after wounding. The mice are treated with LPS when the mice have recovered from anesthesia. Next, 2, 4, 8, 16, and 32 hours later, the corneas of the mice are photographed with the dissecting microscope as above. Tear fluid washings are collected by instilling 1.5 μl of PBS containing 0.1% bovine serum albumin into the conjunctival sac (Luo, L. et al., Invest Ophthalmol Vis Sci, 45:4293-4301, 2004), and the tear fluid and buffer are collected with a 1 μl volume glass capillary tube from the tear meniscus in the lateral canthus, diluted in 30 μl PBS, and used for determination of cytokines by a custom panel of multiplex immunobeads for TNFα, IL-1β, IL-6, MIP-2, and KC (Millipore-Upstate) (Corrales, R. M. et al., Cornea, 26:579-584, 2007). Results are read.

Time points for measuring therapeutic effects: Mice are treated 1-3 times daily (depending on the results from the time-course above) with vehicle or unlabeled TBP1_{COL} for 1, 2, 3, and 5 days. Tear fluid is collected and analyzed as above, and the sizes of the wounds are monitored by photographing after staining with fluorescein. At each time point, corneas are harvested from two TBP1_{COL}-treated and two control mice, homogenized with a manual microhomogenizer (Staples, E. et al., J Immunol Methods, 394:1-9, 2013) in 200 μl tissue protein extraction reagent (TPER, Pierce Bioscience) supplemented with protease inhibitors, clarified by centrifugation, and the cytokine profile is analyzed as above. Parts of the samples are also used for Western Blotting with anti-TBP1 antibodies.

Determining the therapeutic effects: The experimental groups are (5 mice each): (1) no wound, (2) PBS alone, no LPS (3) LPS, (4) LPS+TBP1 without an anchor domain (R&D Systems), (5) LPS+unlabeled TBP1_{COL}, and (6) LPS+0.1% dexamethasone, all in endotoxin-free PBS. The mice are euthanized, and the read-outs are: a) concentrations of cytokines in tear washings, b) sizes of wounds as detected by staining with fluorescein (to ensure wounds are present and collagen is accessible for binding to TBP1_{COL}), c) cytokines and myeloperoxidase concentrations in corneal extracts determined by ELISA (HyCult Biotech) as a measure of influx of neutrophils (Lee, R. H. et al., Proc Natl Acad Sci USA, 111:16766-16771, 2014). In addition, a limited histological analysis is used as follows. Mice are treated as in (3)-(5) (2 mice each group), and sections of the corneas are immunostained with antibodies to collagen I or IV, simultaneously staining with antibodies to TBP1 (Santa Cruz Biotechnology) to localize TBP1_{COL} and with DAPI as a counter-stain.

Protocol: Induction of inflammation in the cornea with LPS always requires some form of wounding, and a wounding model is used (Basu, S. et al., Sci Transl Med, 6:266ra172, 2014). The basement membrane is penetrated, and the wounds take 4-6 days to heal (Basu, S. et al., Sci Transl Med, 6:266ra172, 2014), which provides the experimental advantage that anchor domains that bind to collagen I in the stroma and/or collagen IV in the basement are analyzed in these and future experiments. LPS from P. aeruginosa is used because it is a clinically important pathogen associated with bacterial keratitis (Ong, H. S. et al., Postgrad Med J, 91:565-571, 2015; Fleiszig, S. M. et al., Clin Exp Optom, 85:271-278, 2002; Pereira, R., et al., Drug Dev Res, 76:419-427, 2015), and P. aeruginosa often causes deep corneal ulcers (Tallab, R. T. et al., Br J Ophthalmol, 100:731-735, 2016; Palioura, S. et al., Clin Ophthalmol, 10:179-186, 2016; Hazlett, L. D., Chem Immunol Allergy, 92:185-194, 2007).

The read-outs are important parameters that characterize inflammation. Measurements of cytokines in tears do not require sacrifice of the mice, but the amount of material is limited. Significant measurements in tears have been reported for TNFα, IL-1β, and IL-6 with the Millipore-Upstate reagents used here (Corrales, R. M. et al., Cornea, 26:579-584, 2007; Wei, Y. et al., Invest Ophthalmol Vis Sci, 52:4780-4788, 2011). Influx of neutrophils, which is a hallmark of corneal inflammation induced by LPS (Sonoda, K. H. et al., Cornea, 24:S50-S54, 2005; Pearlman, E. et al., Int Rev Immunol, 32:4-18, 2013), is quantitated by measuring myeloperoxidase. MIP-2 and KC are chemokines that are highly upregulated in the cornea by LPS and have major roles in recruitment of neutrophils (Johnson, A. C. et al., Invest Ophthalmol Vis Sci, 46:589-595, 2005). Dexamethasone will be used as a standard-of-care positive control for treating bacterial-induced inflammation (Tallab, R. T. et al., Br J Ophthalmol, 100:731-735, 2016; Palioura, S. et al., Clin Ophthalmol, 10:179-186, 2016; Hazlett, L. D., Chem Immunol Allergy, 92:185-194, 2007).

Six-eight-week-old C57BL/6J mice are used as originally reported for the wounding model (Basu, S. et al., Sci Transl Med, 6:266ra172, 2014). This mouse strain responds strongly to infection with P. aeruginosa by induction of perforating ulcers in part mediated by LPS (Hazlett, L. D. et al., Invest Ophthalmol Vis Sci, 41:805-810, 2000). Male mice are avoided because the innate immune response in the cornea is strongly influenced by social stress (Dong-Newsom, P. et al., Brain Behav Immun, 24:273-280, 2010), which is less of a concern with female mice. The size of the groups for the final experiment was determined by power analysis of the critical comparison of influx of neutrophils in the groups treated with LPS and LPS and TBP1_{COL} (groups 3 and 5). The assumptions were a minimal effect size of a halving of the measured myeloperoxidase, a standard deviation of 0.4, derived from a study of mice wounded similarly and treated with LPS (Vij, N. et al., Invest Ophthalmol Vis Sci, 46:88-95, 2005), α=0.05 and a power of 0.9 using the unpaired t-test (PS software) (Dupont, W. D. et al., Control Clin Trials, 19:589-601, 1998). The data are analyzed by one-way analysis of variance followed by Tukey post-hoc tests of relevant comparisons.

Results: Treatment with the non-anchored construct will have little to no effect compared to the control (group 3 vs. group 4), whereas treatment with the anchored TBP1_{COL} construct have much larger effects (group 3 vs. group 5). The most direct measure is removal of TNFα from the tear film by TBP1_{COL}. Blocking TNFα in LPS-treated corneas reduces inflammation. TNFα, IL-1β, and IL-6 are interrelated cytokines, and blocking TNFα has been shown to reduce expression of IL-1β, and IL-6 and reduce neutrophil influx in inflamed corneas (Ji, Y. W. et al., Invest Ophthalmol Vis Sci, 54:7557-7566, 2013).

### Example 3

This example discloses using IL-10 attached to anchor domains to block biological effects in vitro. The IL-10 bound to an anchor domain is a signaling molecule that binds collagen to create a prolonged-release system. A fusion of the potent anti-inflammatory cytokine IL-10 (Sabat, R. et al., Cytokine Growth Factor Rev, 21:331-344, 2010) with an anchor domain (IL-10_{COL}) that binds collagen is created; after binding, it is slowly released by dissociation and/or by proteolysis of collagen.

Construction and purification: Two constructs are created with human IL-10 (which is active on mouse cells) (Liu, Y. et al., J Immunol, 152:1821-1829, 1994): two with the collagen-binding anchor domain used above at either terminus connected through GGGGSGGGGS linkers. The constructs also contain a His₆ tag, and the proteins are purified from crude extracts by nickel-chelate chromatography using COMPLETE™ Purification Resin according to the manufacturer's instructions (Roche Life Science), as above (Example 2).

Measurement of activity: Activities are assayed by co-stimulation of growth of MC/9 cells with IL-4 as a co-factor (Doll, F. et al., Arthritis Res Ther, 15:R138, 2013), and potencies are compared to commercial recombinant IL-10 (R&D Systems).

Required dose: The chosen constructs are labeled with ALEXA FLUOR® 488 (see Example 9), and the dose is titrated as in Example 2.

Rationale: IL-10 has a potent anti-inflammatory role in diverse inflammatory processes, and local administration of IL-10 can act as a potent therapeutic (Sabat, R. et al., Cytokine Growth Factor Rev, 21:331-344, 2010; Saxena, A. et al., Cytokine, 74:27-34, 2015). It has been tagged at either terminus with retention of activity (Guo, Y. et al., Protein Expr Purif, 83:152-156, 2012; Doll, F. et al, Arthritis Res Ther, 15:R138, 2013; Takagi, J. et al., Biochemistry, 31:8530-8534, 1992). II-10 has previously been shown to exert effects in the cornea as delivery of IL-10 by gene transfer prolongs survival time of corneal allografts (Saxena, A. et al., Cytokine, 74:27-34, 2015; Klebe, S. et al., Transplantation, 71:1214-1220, 2001).

Results: The biological activity of the construct is assessed.

### Example 4

This example discloses using IL-10 attached to anchor domains to block inflammation in vivo.

Effect of anchored IL-10 constructs in vivo: Although anchored IL-10 counters inflammation by mechanisms that are in principle different from the anchored TBP1 used in Examples 1 and 2, the experiments are similar. The length of time that the two constructs are present and active is first assayed.

Results: The read-outs are identical to Example 2, and the criterion for success is the same.

### Example 5

This example describes creating a fusion protein to manage inflammation in the cornea. An adaptor protein consisting of an anchor domain and a domain that binds antibodies (from protein A, G, or L) is created. The adaptors are tested in the corneas of mice treated with lipopolysaccharide, which is produced by many pathogenic bacteria to cause severe inflammation in the eye (Willcox, M. D., Optom Vis Sci, 84:273-278, 2007). Any antibody can be attached to the surface of the eye using the adaptors. Antibodies to proteins that cause inflammation in the eye (that specifically bind to tumor necrosis factor-α, and interleukin-1β and 6) are used (Ji, Y. W. et al., Invest Ophthalmol Vis Sci, 54:7557-7566, 2013; Sakimoto, T. et al., Anti-inflammatory effect of IL-6 receptor blockade in corneal alkali burn, Exp Eye Res, 97:98-104, 2012; Okanobo, A. et al., Am J Ophthalmol, 154:63-71, 2012). The following groups are compared: 1) no treatment, 2) a steroid (the best current treatment option) 3) the adaptor alone 4) each of the antibodies alone, and 5) antibodies with the adaptor. The outcome is measured with a battery of parameters that are conventionally used to monitor inflammation. The free antibodies without the adaptor have minor or no effects, whereas immobilized antibodies significantly reduce inflammation.

### Example 6

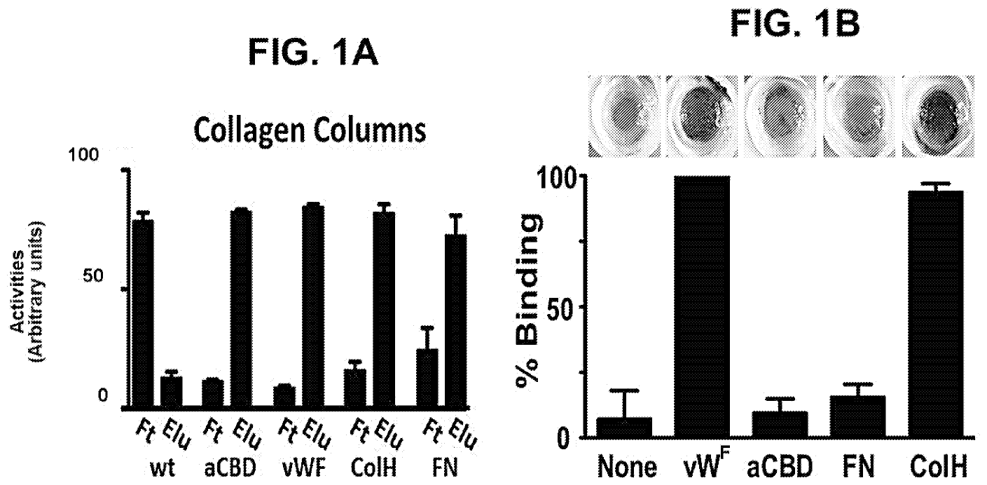

This example discloses that anchor domains can be used to attach heterologous proteins rapidly to surfaces of eyes. For FIG. 1A, fusion proteins were produced using GGGGS linkers (SEQ ID NO: 9) with the sequence WREPSFMALS (SEQ ID NO: 1) from the von Willebrand factor (Andrades, J. A. et al., Exp Cell Res, 250:485-498, 1999), the 20 kDa-binding domain from Clostridium collagenase (Nishi, N. et al., Proc Natl Acad Sci USA, 95:7018-7023, 1998), a collagen-binding domain from a collagenase from Clostridium histolyticum (Uchida, K. et al. Acceleration of periosteal bone formation by human basic fibroblast growth factor containing a collagen-binding domain from Clostridium histolyticum collagenase. J Biomed Mater Res A 102, 1737-1743, doi:10.1002/jbm.a.34841 (2014)), an artificial collagen-binding domain (de Souza, S. J. & Brentani, R. Collagen binding site in collagenase can be determined using the concept of sense-antisense peptide interactions. J Biol Chem 267, 13763-13767 (1992)), or the HS-binding domain (SEQ ID 4). The wt is a control with no anchor domain. The proteins also contained a His₆ tag. Crude extracts were made using BPER™ reagent (ThermoFisher Scientific) and sonication, and the proteins were purified by nickel-chelate chromatography using COMPLETE™ His-Tag Purification Resin according to the manufacturer's instructions (ROCHE® Life Science). The proteins were transferred to phosphate buffered saline (PBS) using AMICON® Ultra Centrifugal Filters (MILLIPORE®). The purified proteins were applied to 200 μl (approximately) columns of heparin SEPHAROSE™ or collagen III agarose (SIGMA-ALDRICH™) in PBS with 50 μg/ml bovine serum albumin (BSA) as a carrier. The columns were washed and eluted with the same buffer containing 2 M NaCl and 0.5% Triton X-100. LacZ activities in the fractions were assayed by a standard colorimetric assay using ortho-nitrophenyl-β-galactoside, and the measurements were adjusted for the effects of the high salt on column flow through (Ft) and eluate (Elu) fraction activity measurements. The data are the means of four determinations, and the error bars are standard deviations.

For FIG. 1B, rabbit eyes (PELFREEZ BIOLOGICALS™) were wounded by scraping with sandpaper, which exposes the collagen in the interior part of the cornea (the “stroma”). Equal amounts of the LacZ fusion proteins, as determined by enzymatic activity, were applied to the eyes for two minutes in 2 μl PBS, the eyes were washed, and the proteins were detected with X-gal (top row), which turns blue in the presence of LacZ. The eyes were photographed, and the staining intensities were determined using the ImageJ program. The values are the means of 4 determinations, and the error bars are standard deviations.

Discussion: The data show that the vWF factor and the ColH anchor domains can mediate rapid binding to collagen in the cornea. The inability of some potential anchor domains (aCBD and FN) to bind to eyes may be due to slow binding kinetics and/or because some potential binding sites are masked, for instance by collagen-binding molecules.

### Example 7

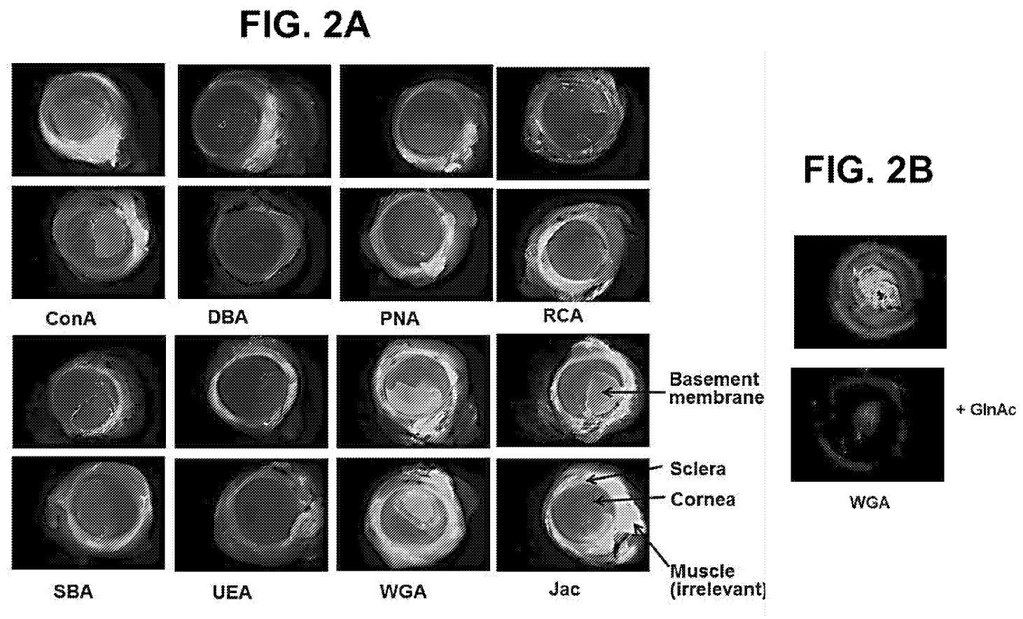

This example discloses lectins that bind rapidly to the surface of eyes. For FIG. 2A, equal amounts of fluorescein-labeled lectins (VECTOR® Labs) in 10 μl PBS with 0.1% BSA were applied to the surface of rabbit eyes for two minutes, and the eyes were washed and photographed using a fluorescence stereomicroscope. The results from two experiments are shown. The eyes had been exposed to Pseudomonas aeruginosa the day before, which induced lesions in many of the eyes. For FIG. 2B, the eyes were incubated as indicated in FIG. 2A in the presence or absence of competitive inhibitor 0.2 M GlnAc, a competitor for binding. One of duplicate samples are shown.

Discussion: The analysis was performed because the literature on WGA binding to the ocular surface is confusing. Gipson et al. (Dev Biol, 96: 337-345, 1983) reported that there is no WGA reactivity in the superficial epithelial cells of the cornea, implying that topically applied fusion proteins with the WGA anchor domain cannot bind to the cornea. However, Holmes et al. (Cornea, 4: 30-34, 1985) and Wells et al. (Exp Eye Res, 46: 485-497, 1988) reported WGA reactivity in these cells. Tuori et al. (Histochem J, 26: 787-798, 1994) found weak WGA reactivity in the cornea among a battery of 11 lectins. All of these studies were performed using histochemical techniques with long exposures to the lectins at equilibrium or near-equilibrium conditions, which does not allow for an evaluation of binding for topically administered lectins after a short exposure. Chen et al. analyzed binding lectins to non-fixed corneal surface in organ culture and reported minimal binding of WGA relative to another lectin (concanavalin A). Mochizuki et al. (Clin Ophthalmol, 4: 925-930, 2010) studied the structure of the glycocalyx at the eye surface in human volunteers and reported binding of WGA, but no comparison with other lectins was provided. Therefore, it has not been possible to predict which lectin(s) would be useful anchors for therapeutic proteins when applied to the cornea as eye drops.

These experiments show that a brief exposure to WGA yields maximal attachment among the lectins tested. Notably, WGA binds both to the surfaces of the cornea and the sclera and binds strongly to wounded regions. Jacalin and ConA bind to a significant extent.

### Example 8

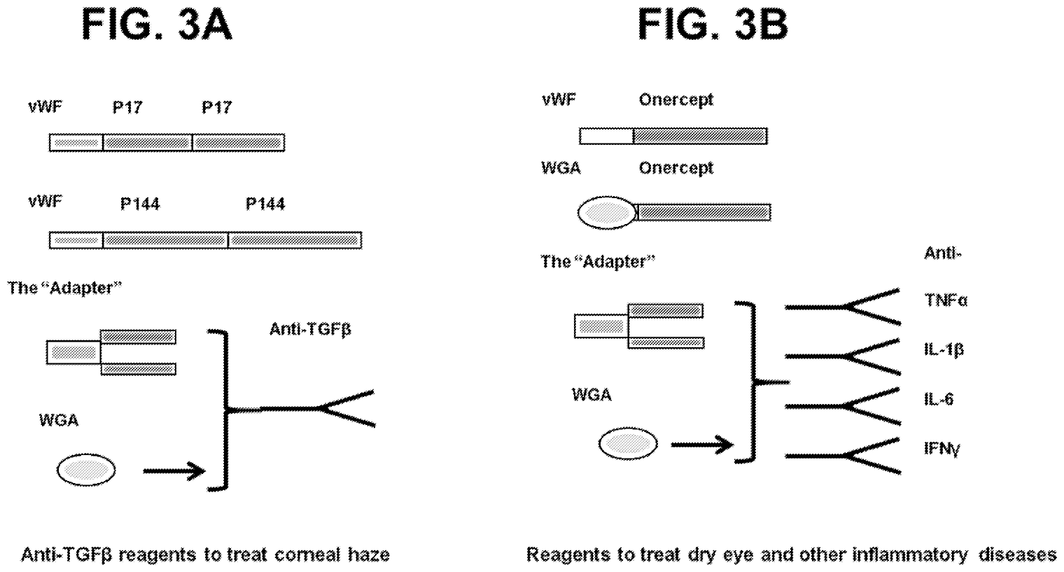

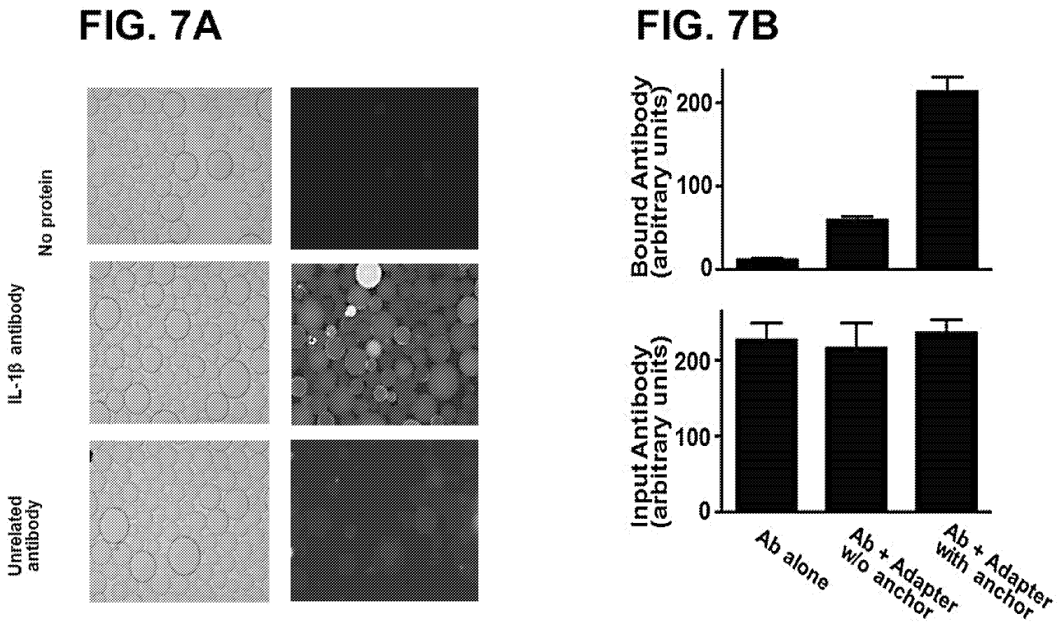

This example discloses compounds that have been produced for treating eye diseases. FIG. 3A shows reagents that can be used to block the activity of TGFβ to prevent haze, as follows (from top to bottom): two blocking peptides, P17 and P144 (Dotor et al., Cytokine 39: 106-115, 2007; Ezquerro et al., Cytokine, 22: 12-20, 2003) fused to the vWF anchor domain via linkers: GGGGSGGGGSEAAAK for P144 and GGGGSGGGGSGGGGS for P17. The P17 and P144 peptides are duplicated to maximize TGFP binding. Between the two P17 domains is the linker GGGGSGGGGS, and between the two P144 domains is the linker EAAAKEAAAK. The “adaptor” consists of the vWF anchor domain coupled via a GGGGSGGGGSAS linker to the antibody-binding domain of protein G (see also FIG. 7B), which binds a large repertoire of antibodies, including anti-TGFP antibodies, via non-covalent interactions. Therefore, this adaptor can serve as a general molecular device for immobilizing antibodies to collagen. Alternatively, covalent conjugation of antibodies to WGA provides an anchor that allows for binding to the surface of the eye (see FIGS. 2A-2B). FIG. 3B shows reagents that can be used to block inflammation, which is fundamental for the pathogenesis of dry eye (Bron et al., Ocul Surf, 15: 438-510, 2017). From top to bottom, FIG. 3B shows the following: anchored forms of tumor necrosis factor-α binding protein 1 (TBP1; also known as ONERCEPT®) and antibodies to TNFα, IL-1, IL-6, and INFγ that are anchored either via the adapter or by covalent conjugation of WGA.

### Example 9

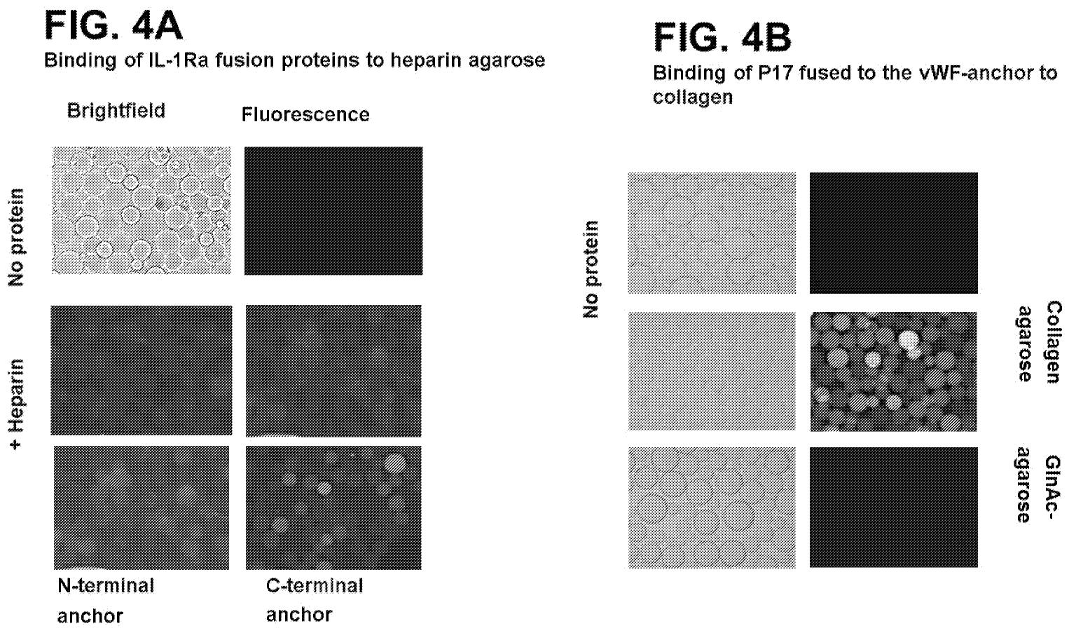

This example illustrates that HS- and vWF-derived anchors confer binding activities of therapeutic proteins to their cognate ligands. In the experiments illustrated in FIG. 4A, approximately 10 μl of heparin-conjugated agarose beads (SIGMA-ALDRICH™) was incubated with no protein or with ALEXA FLUOR® 488-labeled IL-1Ra. Labeling was performed by incubating the purified proteins in 100 mM sodium carbonate, pH 8.9, for 1 hour at room temperature with 0.05-0.25 mg/ml ALEXA FLUOR® 488 carboxylic acid, 2,3,5,6-tetrafluorophenyl ester (MOLECULAR PROBES®). Excess fluorophore was removed, and the protein transferred to PBS by centrifugation in AMICON® Ultra Centrifugal Filters, 10 kDa cut-off (MILLIPORE®). Equal amounts of the Anakinra tagged at either terminus was incubated with the beads for 20 minutes at room temperature. Where indicated, heparin (at approximately 1 mg/ml) was included as a competitor. In FIG. 4B, P17 tagged with the vWF anchor domain was labeled with ALEXA FLUOR® 488 in the same manner as the IL-1Ra (above). Further, 10 μl collagen III agarose (SIGMA-ALDRICH™) was incubated with no protein or with the labeled P17 for 20 minutes at room temperature. As a control, the protein was also incubated with agarose attached to an unrelated ligand, GlnAc (SIGMA-ALDRICH™).

Discussion: The data demonstrate that these pharmacologically actively proteins and peptides can be attached to anchor domains such that they can bind to their cognate ligands.

### Example 10

This example illustrates that WGA can be covalently bound to antibodies while maintaining the binding activity of the lectin. To cross-link WGA, 100-250 μg WGA was placed in a 0.5 ml AMICON® MW centrifugation device, 10 kDa cut-off, and transferred to 50 mM 2-morpholinoethanesulfonic acid; 2-(4-morpholino)ethanesulfonic acid (MES), 500 mM NaCl, pH 6.0 (buffer 1) for a final volume of 50-100 μl . Further, 20 mM EDC (1-ethyl-3-(3-dimethylaminopropyl)carbodiimide hydrochloride) and 100 mM Sulfo-NHS (N-hydroxysulfosuccinimide) were dissolved in water and immediately mixed with the WGA solution at a 1/10 volume and incubated for 10 minutes at room temperature. The reaction was washed 2 times with buffer 1 in the centrifugation devices and then washed once more with 0.1 M sodium phosphate, pH 7.5. The antibody was then added in a minimal volume of PBS. The reaction proceeded for 2 hours at room temperature and was then quenched by adding 10 μl 100 mM ethanolamine. The reaction was then transferred to 100 kDa AMICON® centrifugation devices and washed with PBS to remove WGA monomers and dimers and excess ethanolamine.

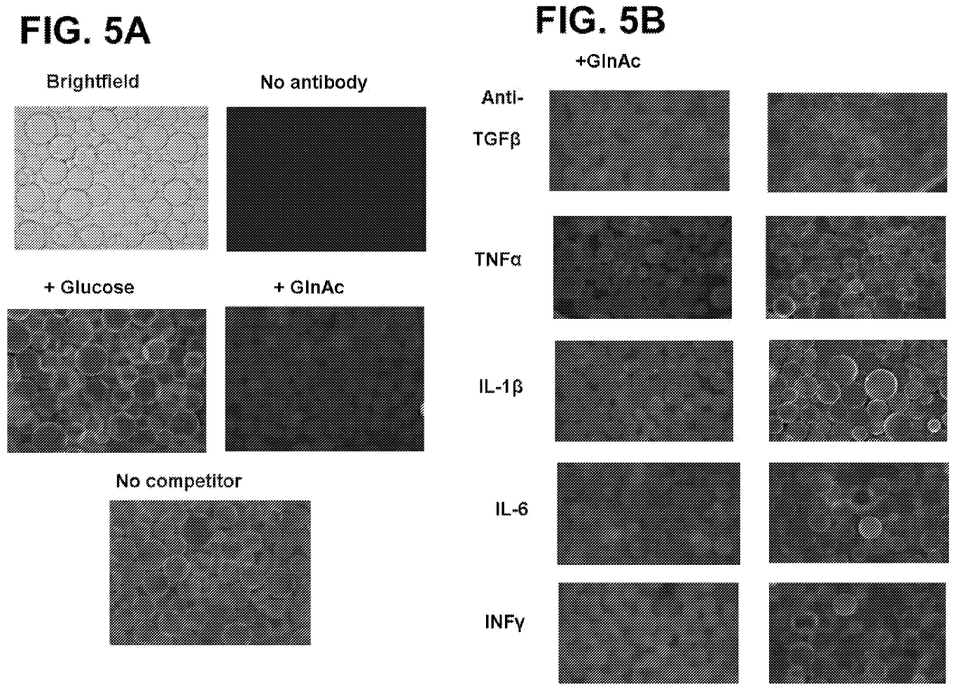

FIG. 5A shows the binding properties of a WGA-conjugate of a commercial Alexa Fluor® 488-labeled goat anti-human IgG (INVITROGEN®) to GlnAc agarose beads. The top photographs show background fluorescence of the beads alone. The lower three photographs show beads incubated with WGA-conjugated antibody in the presence of 0.5 M glucose (which does not compete for binding), 0.5 M GlnAc (which competes for binding), or with no addition.

FIG. 5B shows the binding of neutralizing antibodies to inflammatory cytokines, as determined using experiments performed similar to the experiments used to generate the data in FIG. 5A. The data from top to bottom are as follows: the 1D11 anti-TGFβ1 antibody (INVITROGEN®), the MM425B anti-IL-1β antibody (INVITROGEN®), the MP6-XT22 anti-TNFα antibody (INVITROGEN®), the anti-IL-6 MP5-20F3 antibody (R&D SYSTEMS®), and the H22 anti-INFγ antibody (R&D SYSTEMS®).

Discussion: The data demonstrate that the coupling procedure for WGA is versatile and that the conjugates have strong and specific binding activity to GlnAc. FIG. 5B shows that antibodies to five different targets of therapeutic value can be conjugated to WGA such that the conjugate retains the ability to bind to the sugar ligand. Note that the beads have some autofluorescence, and binding appears as a “ring” around the beads.

### Example 11

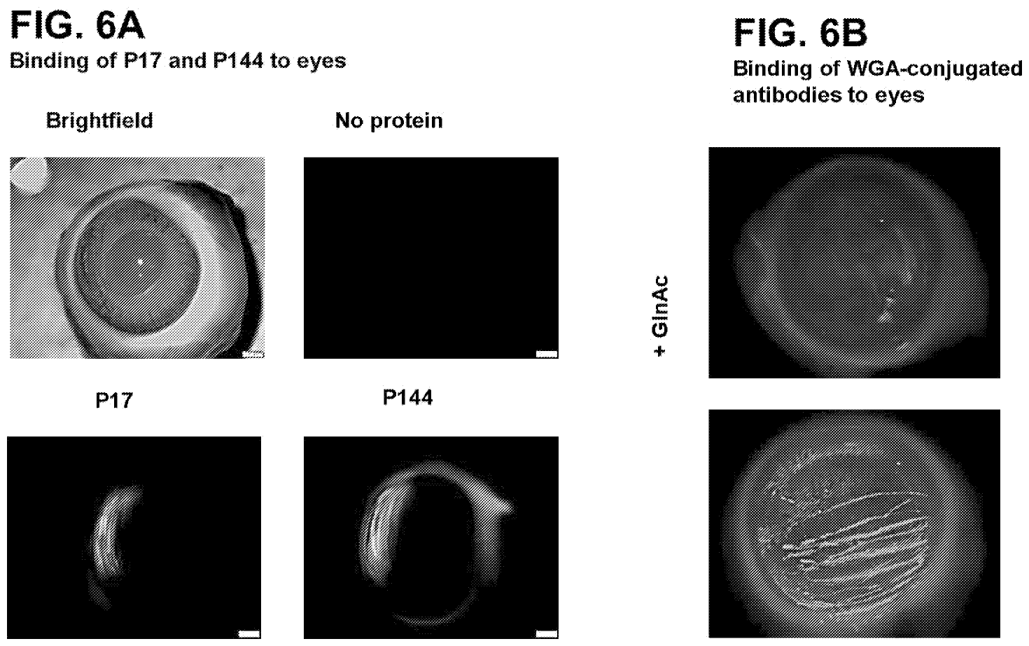

This example discloses the use of anchor domains to bind topically applied therapeutic proteins to the surface of eyes. FIG. 6A shows rabbit eyes that were wounded ex vivo with 30-40 scratches into the stroma (the interior part of the cornea) using a 20-gauge needle. The eyes were then incubated with P17 or with P144 attached to a vWF anchor and labeled with ALEXA FLUOR™ 488 (see Example 9). The proteins were incubated for 2 minutes in 10 μl PBS with 0.1% BSA. FIG. 6B shows rabbit eyes that were wounded in a similar manner as in FIG. 6A and incubated with ALEXA FLUOR™ 488-labeled goat anti-human IgG (INVITROGEN®). The top sample additionally contained 0.5 mM GlnAc as a competitor for binding.

Discussion: These data reveal that therapeutic proteins can bind the eye surface when attached to anchor domains. The vWF binds strongly to the wounds in the cornea, as expected due to its high collagen concentration (Hassell et al., Exp Eye Res, 91: 326-335, 2010).

### Example 12

This example discloses that therapeutic proteins can be attached to anchors while maintaining their biological and biochemical activities. For FIG. 7A, 10 μl GlnAc agarose beads (SIGMA®) were incubated with unlabeled MM425B anti-IL-1β antibody conjugated to WGA (see Example 15) or with an unrelated antibody conjugated to WGA (the anti-TGFβ1 1D11 antibody (INVITROGEN®)) for 20 minutes, washed 3 times with PBS, and incubated with IL-1β that was labeled with ALEXA FLUOR® 488 (see Example9) in 5 μl PBS with 0.1% BSA with occasional shaking for 20 minutes. The beads were washed with 0.5 ml PBS 2 times and photographed with a fluorescence microscope. For FIG. 7B, alkaline phosphatase conjugated goat anti-human IgG (Invitrogen) was incubated with a 10-fold molar excess of the adaptor with or without the anchor domain in 30 μl PBS with 0.1% BSA for 2 hours. Free adaptor was removed by centrifugation in 100 kDa cut-off AMICON® Ultra Centrifugal Filter devices (MILLIPORE®) and washing with PBS with 0.1% BSA. The samples were diluted to 50 μl in the same buffer, and the same amounts of the adapter with and without anchor were incubated in wells of collagen I-coated BIOCOAT® 96 well plates (CORNING®) for two hours with constant agitation. After extensive washing, the bound antibody was detected using a standard colorimetric assay with para-nitrophenylphosphate. The upper graphs show the activities bound to the collagen coated plates, and the lower graph shows the input activities.

Discussion: The experiments show that the binding activity of antibodies is retained while conjugated to WGA. The adapter can be used as a general means of attaching antibodies to collagen.

### Example 13

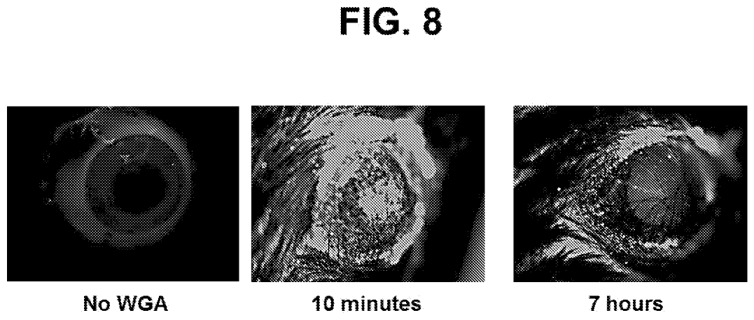

This example discloses that WGA remains on the eye surface of for a prolonged period of time. For FIG. 8, seven-week-old female C57BL/c underwent operations to remove the lachrymal gland, as described (Stevenson et al., Cornea, 33: 1336-1341, 2014) 11 days before sacrifice. One microliter of fluorescein-labeled WGA at 2 mg/ml (VECTOR® Laboratories) was applied to eyes for 2 minutes, and the mice were sacrificed 10 minutes or 7 hours later. The regions around the eyes were washed with PHB to remove the significant amounts of the attached conjugated lectin, which interfered with visualization. The eyes were photographed using a fluorescence stereomicroscope.

Discussion: The data show that WGA is a useful anchor for promoting prolonged residency times at the eye surface.

### Example 14

This example discloses using a TGFβ-blocking agent (e.g., the TGFβ-blocking agents shown in FIG. 3A) to block formation of haze in vivo.

The mouse model of corneal haze was developed and has been used extensively to analyze effects of stem cell transplantation (Du et al., Stem Cells, 27: 1635-1642, 2009; Basu et al., Sci Transl Med, 6: 266ra172, 2014). To induce haze, corneas of mice are wounded through the basement membrane with an instrument that was originally designed to remove rust using a rotating burr (ALGERBRUSH® IIR; Stepp et al., Exp Eye Res, 121: 178-193, 2014). Haze is clearly apparent two weeks after the operation and is associated with a massive development of myofibroblasts, which is similar to late haze induced by refractive surgeries (Torricelli et al., Exp Eye Res, 142: 110-118, 2016; Tomas-Juan et al., J Optom, 8: 149-169, 2015).

The dose for optimal binding is first determined using fluorescently labeled compounds (see Example 14) at increasing doses, for instance 1, 3.3, 10, 33, 100, 330, and 1000 ng/ml in PBS using 1-2 μl per eye, and then photographing and quantitating the bound fluorescence as in Example2.

Mice are then treated with the optimal dose of the fluorescently labeled compound, and the retention of the fluorescence is monitored over time, for instance after 15 minutes, 1, 3, 8, 24, 48, and 96 hours. Some mice are harvested and the corneas used for immunoblotting with relevant antibodies to determine the integrity of the proteins.

To determine the effects on haze, the mice are treated with the compounds at the optimal dose. The frequency of treatment depends on the results of the retention time course. In some examples, the mice are treated twice daily, which is compatible with a treatment regimen for human patients. Treatment begins after the mice have recovered from the anesthesia and continues until the wounds are closed (1-3 days after the operation), which is determined by staining with fluorescein or Lissamine Green. The treatment groups (6 mice each) include a mock treatment and treatment with each of the compounds. Treatment is followed by a similar experiment, which compares the mock treatment and treatment with the compound(s) with and without anchor domains. Compounds with the anchor are expected to exert a much greater effect than the corresponding compounds without the anchor domain.

Two and four weeks after corneal debridement, the eyes are collected, and the entire globes are imaged using a dissecting microscope with indirect illumination (Hertsenberg et al., PLoS One, 12: e0171712, 2017). Images of the corneas are captured with a camera, and the areas of the eye images containing haze are determined by a masked observer using ImageJ software. Statistical analysis of the values is performed with Prism 7 (GRAPHPAD PRISM®) using ANOVA and the Bonferini correction. Fibrotic marker expression is measured by qPCR using primers that detect smooth muscle actin, collagen type III, and tenascin c (Hertsenberg et al., PLoS One, 12: e0171712, 2017), and the presence of myofibroblasts is determined by staining histological slides with anti-smooth muscle actin using standard techniques.

These experiments assay compounds for treating wounding-induced corneal haze.

### Example 15

This example discloses using compounds (e.g., the dry eye-blocking agents of FIG. 3B) to block dry eye. Dry eye is fundamentally an autoimmune disease, and the reagents are selected to block important inflammatory cytokines (Bron et al., Ocul Surf, 15: 438-510, 2017).

Two mouse models are used. In one, the lachrymal gland is removed by surgical excision (Stevenson et al., Cornea, 33: 1336-1341, 2014), and the other model is an autoimmune regulator gene knockout model. Both models have been established as excellent models for dry eye disease (Chen et al., Lab Invest, 92: 556-570, 2012; Stevenson et al., Cornea, 33: 1336-1341, 2014). Treatments begin after the mice have recovered from anesthesia in model 1 and continue for 14 days. The treatments in model 2 begin when the mice are 5-6 weeks old and continue for 14 days. The dose and treatment regiments are similar to the description in Example 14. Further, the treatment groups are analogous, except an additional group is treated with 0.1% dexamethasone solution, as a standard-of-treatment comparison.

Two weeks after the treatments are initiated, the eyes are stained with Lissamine Green or fluorescein to determine the extent of damage to the corneal epithelium (Wolffsohn et al., Ocul Surf, 15: 539-574, 2017). Inflammatory cell infiltration is determined using flow cytometry, in which the corneas are harvested, dissociated with collagenase, and stained with a battery of fluorophore-conjugated antibodies to detect neutrophils, macrophages, and monocytes as well as CD4+, CD8+, and CD45+ cells (Yun et al., J Virol, 88: 7870-7880, 2014). Goblet cell density is a marker of dry eye and is determined using a periodic acid Schiff staining protocol (Zhang et al., Exp Eye Res, 118: 117-124, 2014; Pflugfelder et al., Invest Ophthalmol Vis Sci, 56: 7545-7550, 2015). These experiments assay therapeutic proteins for dry eye treatment.

### Example 16

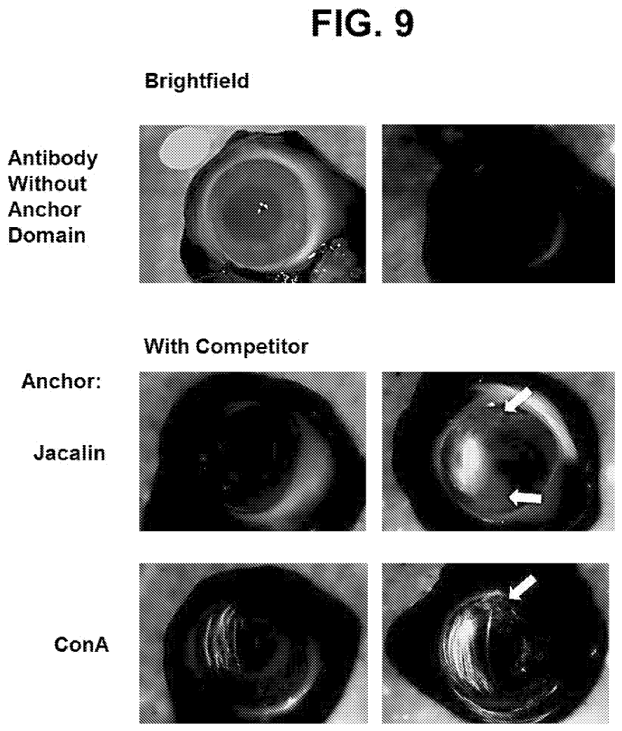

This example illustrates that the lectins jacalin and conA (concanavalinA) can be covalently bound to antibodies while maintaining the binding activity of the lectins. The lectins were cross-linked to a commercially available antibody, ALEXA FLUOR® 488-labeled goat anti-human IgG (INVITROGEN®), as in Example 10 and incubated as in Example 11 on rabbit eyes that had been scratched with a needle. The eyes used in this example were frozen, and the eyes show binding from remnants of the epithelium (white arrows) to the interior of the scratches (FIG. 9). For a competition experiment, the competitor 0.2 M galactose (jacalin) or 0.2 M methyl α-D-mannopyranoside (conA) was added (FIG. 9).

Discussion: The data demonstrate that the coupling procedure for lectins can be used for jacalin and conA, and the resulting conjugates bind tissues in the eye. The binding is specific because it shows competition with the cognate ligand. Therefore, conjugating antibodies to lectins is a universal procedure for binding antibodies to tissues in the eye.

1. A fusion protein comprising an anchor domain and a therapeutic polypeptide, wherein:
(1) the anchor domain comprises a lectin carbohydrate-binding anchor domain, a von Willebrand factor (vWF) collagen-binding anchor domain, a Clostridium collagenase (ColH) collagen-binding anchor domain, or a heparin-binding (HS) anchor domain, and the therapeutic polypeptide comprises a transforming growth factor beta (TGFβ) antagonist, a tumor necrosis factor alpha (TNFα) antagonist, an interleukin (IL)-1β antagonist, an IL-6 antagonist, an immunoglobulin-binding (Ig) polypeptide, or an interferon (IFN)γ antagonist; or
(2) the anchor domain comprises a lectin carbohydrate-binding anchor domain, a von Willebrand factor (vWF) collagen-binding anchor domain, or a Clostridium collagenase (ColH) collagen-binding anchor domain, and
the therapeutic polypeptide comprises IL-10 or interleukin-1 receptor antagonist (IL-1Ra).

2. The fusion protein of claim 1 wherein the lectin carbohydrate-binding anchor domain is a wheat germ agglutinin (WGA), jacalin (Jac), or concanavalin A (conA) carbohydrate-binding anchor domain.

3. The fusion protein of claim 1, wherein:
the TGFβ antagonist comprises P17, P144, or an antibody that specifically binds TGFβ;
the TNFα antagonist comprises an inhibitor of TNFα polypeptide from the TNF receptor 1 or an antibody that specifically binds TNFα;
the IL-1β antagonist comprises an antibody that specifically binds IL-1β;
the IL-6 antagonist comprises an antibody that specifically binds IL-6;
the IFNγ antagonist comprises an antibody that specifically binds IFNγ; and/or
the Ig-binding polypeptide comprises protein A, protein G, or protein L or Ig-binding fragment thereof.

4. The fusion protein of claim 3, wherein:
(1) the anchor domain comprises a lectin carbohydrate-binding anchor domain, and the therapeutic polypeptide comprises the inhibitor of TNFα polypeptide from the TNF receptor 1, IL-10, IL-1Ra, or an antibody that specifically binds TGFβ, TNFα, IL-1β, IL-6, or IFNγ;
(2) the anchor domain comprises the vWF collagen-binding anchor domain or the ColH collagen-binding anchor domain, and the therapeutic polypeptide comprises the inhibitor of TNFα polypeptide from the TNF receptor 1, IL-1Ra, IL-10, P17, P144, or the Ig-binding polypeptide; or
(3) the anchor domain comprises the HS anchor domain, and the therapeutic polypeptide comprises the inhibitor of TNFα polypeptide from the TNF receptor 1.

5. The fusion protein of claim 1, wherein:
the lectin carbohydrate-binding anchor domain comprises the amino acid sequence of SEQ ID NO: 6, SEQ ID NO: 7, or SEQ ID NO: 8;
the vWF collagen-binding anchor domain comprises the amino acid sequence of SEQ ID NO: 1;
the ColH collagen-binding anchor domain comprises the amino acid sequence of SEQ ID NO: 3; and/or
the HS anchor domain comprises the amino acid sequence of SEQ ID NO: 5.

6. The fusion protein of claim 1, wherein:
(1) the anchor domain comprises the vWF collagen-binding anchor domain, and the therapeutic polypeptide comprises P17, P144, or the Ig-binding polypeptide; or
(2) the anchor domain comprises the lectin carbohydrate-binding anchor domain, and the therapeutic polypeptide comprises the antibody that specifically binds TGFβ.

7. The fusion protein of claim 1, wherein:
(1) the anchor domain comprises the vWF collagen-binding anchor domain, and the therapeutic polypeptide comprises the Ig-binding polypeptide, the inhibitor of TNFα polypeptide from the TNF receptor 1, IL-1Ra, or IL-10; or
(2) the anchor domain comprises the lectin carbohydrate-binding anchor domain, and the therapeutic polypeptide comprises the inhibitor of TNFα polypeptide from the TNF receptor 1, IL-10, IL-1Ra, or the antibody that specifically binds TNFα, IL-1β, IL-6, or IFNγ; or
(3) the anchor domain comprises the HS anchor domain, and the therapeutic polypeptide comprises the inhibitor of TNFα polypeptide from the TNF receptor 1.

8. The fusion protein of claim 1, wherein the fusion protein comprises a linker polypeptide between the anchor domain and therapeutic polypeptide.

9. An isolated nucleic acid molecule encoding the fusion protein of claim 1.

10. The isolated nucleic acid molecule of claim 9, operably linked to a heterologous promoter.

11. An expression vector comprising the isolated nucleic acid molecule of claim 10.

12. A method for treating a subject with a disorder that affects the eye, comprising:
(1) administering to the eye of the subject a therapeutically effective amount of the fusion protein of claim 1, wherein the therapeutic polypeptide comprises IL-1Ra, IL-10, P17, P144, the inhibitor of TNFα polypeptide from the TNF receptor 1, or the antibody that specifically binds TGFβ, TNFα, IL-1β, IL-6, or IFNγ; or
(2) administering to the eye of the subject a therapeutically effective amount of:
(a) the fusion protein of claim 1, wherein the therapeutic polypeptide comprises the Ig-binding polypeptide, and
(b) any therapeutically effective antibody,
thereby treating the disorder that affects the eye.

13. The method of claim 12, wherein the subject has corneal haze or corneal scarring, and:
(1) wherein the therapeutic polypetide comprises specifically binds TGFβ; or
(2) wherein the therapeutic polypeptide comprises the Ig-binding polypeptide; and
the therapeutically effective antibody comprises an antibody the specifically binds TGFβ,
thereby treating the corneal haze or corneal scarring.

14. The method of claim 12, wherein the subject has dry eye, and:
(1) wherein the therapeutic polypeptide comprises IL-1Ra, IL-10, the inhibitor of TNFα polypeptide from TNF receptor 1, or an antibody that specifically binds TNFα, IL-1β, IL-6, or IFNγ; or
wherein the therapeutic polypeptide comprises the Ig-binding polypeptide; and
the therapeutically effective antibody comprises an antibody that specifically binds TNFα, IL-1β, IL-6, and IFYγ,
thereby treating the dry eye.

15. The method of claim 14, wherein the subject has Sjögren's syndrome.

16. The method of claim 12, wherein the disorder that affects the eye comprises inflammation at an ocular surface of the eye, and wherein:
(1) the therapeutic polypeptide comprises IL-10, the inhibitor of TNFα polypeptide from TNF receptor 1, IL-1Ra, or the antibody that specifically binds TNFα, IL-1β, IL-6, or IFNγ; or
(2) the therapeutic polypeptide comprises the Ig-binding polypeptide; and
the therupeutically effective antibody comprises antibody that specifically binds TNFα, IL-1β, IL-6, or IFNγ,
thereby treating the inflammation at the ocular surface of the eye.

17. The method of claim 16, wherein the inflammation is caused by an acanthamoebal, a bacterial, a fungal, or a viral infection.

18. The method of claim 17, wherein the inflammation at the ocular surface of the eye is keratitis caused by a bacterial infection.

19. A pharmaceutical composition comprising a therapeutically effective amount of the fusion protein of claim 1 and a pharmaceutically acceptable carrier formulated for external and/or topical administration to the eye.

20-25. (canceled)

26. A unit dose dispenser for dispensing the pharmaceutical composition of claim 19 in metered droplets.


SEQUENCE LISTING (19 sequences)

SEQ ID NO: 1 - protein, 10 aa, Artificial Sequence
  vWF polypeptide
WREPSFMALS

SEQ ID NO: 2 - protein, 10 aa, Artificial Sequence
  vWF polypeptide
WREPSFCALS

SEQ ID NO: 3 - protein, 215 aa, Artificial Sequence
  Clostridium polypeptide
EIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 4 - protein, 21 aa, Artificial Sequence
  HS-binding polypeptide
KRKKKGKGLGKKRDPSLRKYK

SEQ ID NO: 5 - protein, 21 aa, Artificial Sequence
  HS-binding polypeptide
KRKKKGKGLGKKRDPCLRKYK

SEQ ID NO: 6 - protein, 290 aa, Artificial Sequence
  ConA polypeptide
MAISKKSSLFLPIFTFITMFLMVVNKVSSSTHETNALHFMFNQFSKDQKDLILQGDATTGTDGNLELTRVSSNGSPQGSSVGRALFYAPVHIWESSAVVASFEATFTFLIKSPDSHPADGIAFFISNIDSSIPSGSTGRLLGLFPDANVIRNSTTIDFNAAYNADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNEIPDIATVV

SEQ ID NO: 7 - protein, 212 aa, Artificial Sequence
  WGA polypeptide
MKMMSTRALALGAAAVLAFAAATAQAQRCGEQGSNMECPNNLCCSQYGYCGMGGDYCGKGCQNGACWTSKRCGSQAGGATCTNNQCCSQYGYCGFGAEYCGAGCQGGPCRADIKCGSQAGGKLCPNNLCCSQWGFCGLGSEFCGGGCQSGACSTDKPCGKDAGGRVCTNNYCCSKWGSCGIGPGYCGAGCQSGGCDGVFAEAITANSTLLQE

SEQ ID NO: 8 - protein, 217 aa, Artificial Sequence
  Jac polypeptide
MAYSSLLSLSVLALLFSISSADTRKWFLANGINQNPIGIIEAAVGVSEDLLNLNGMEAKNDEQSGISQTVIVGPWGAKVSTSSNGKAFDDGAFTGIREINLSYNKETAIGDFQVVYDLNGSPYVGQNHKSFITGFTPVKISLDFPSEYIMEVSGYTGNVSGYVVVRSLTFKTNKKTYGPYGVTSGTPFNLPIENGLIVGFKGSIGYWLDYFSMYLSL

SEQ ID NO: 9 - protein, 5 aa, Artificial Sequence
  linker
GGGGS

SEQ ID NO: 10 - protein, 116 aa, Artificial Sequence
  Ig-binding polypeptide
ADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPK

SEQ ID NO: 11 - protein, 77 aa, Artificial Sequence
  Ig-binding polypeptide
KTFTVTEKPEVIDASELTPAVTTYKLVINGKTLKGETTTKAVDAETAEKAFKQYANDNGVDGVWTYDDATKTFTVTE

SEQ ID NO: 12 - protein, 161 aa, Artificial Sequence
  TNF receptor 1 polypeptide
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 13 - protein, 14 aa, Artificial Sequence
  P144 polypeptide
TSLDASIIWAMMQN

SEQ ID NO: 14 - protein, 15 aa, Artificial Sequence
  P17 polypeptide
KRIWFIPRSSWYERA

SEQ ID NO: 15 - protein, 176 aa, Artificial Sequence
  Exemplary fusion protein
MWREPSFMALSASGGGGSGGGGSASMGTPAVTTYKLVINGKTLKGETTTKAVDAETAEKAFKQYANDNGVDGVWTYDDATKTFTVTEVNTPAVTTYKLVINGKTLKGETTTKAVDAETAEKAFKQYANDNGVDGVWTYDDATKTFTVTEIGENLYFQGIDENLYFQGGSHHHHHHH

SEQ ID NO: 16 - protein, 186 aa, Artificial Sequence
  Exemplary fusion protein
MSKKHHHHHHHHGGGGSASMGTPAVTTYKLVINGKTLKGETTTKAVDAETAEKAFKQYANDNGVDGVWTYDDATKTFTVTEVNTPAVTTYKLVINGKTLKGETTTKAVDAETAEKAFKQYANDNGVDGVWTYDDATKTFTVTEIGENLYFQGIDENLYFQGGSAGGGGSGGGGSASWREPSFMALS

SEQ ID NO: 17 - protein, 82 aa, Artificial Sequence
  Exemplary fusion protein
MSKKWREPSFMALSASGGGGSGGGGSEAAAKTSLDASIIWAMMQNEAAAKEAAAKTSLDASIIWAMMQNGGGGSHHHHHHHH

SEQ ID NO: 18 - protein, 84 aa, Artificial Sequence
  Exemplary fusion protein
MSKKWREPSFMALSASGGGGSGGGGSGGGGSKRIWFIPRSSWYERAGGGGSGGGGSKRIWFIPRSSWYERAGGGGSHHHHHHHH

SEQ ID NO: 19 - protein, 10 aa, Artificial Sequence
  linker
EAAAKEAAAK